(19) United States
(12) Patent Application Publication
(10) Pub. No.: US 2004/0121994 A1
(43) Pub. Date: Jun. 24, 2004

(54) Novel amides that activate soluble guanylate cyclase

(76) Inventors: Steven N. Anderson, Aurora, IL (US); Pramila Bhatia, Libertyville, IL (US); Teodozyj Kolasa, Lake Villa, IL (US); Masaki Nakane, Lake Bluff, IL (US); Meena V. Patel, Green Oaks, IL (US); Jeffrey J. Rohde, Evanston, IL (US); Zhiren Xia, Wadsworth, IL (US); Henry Q. Zhang, Grayslake, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/325,297

(22) Filed: Dec. 20, 2002

(51) Int. Cl. A61K 31/55; A61K 31/541; A61K 31/5377; A61K 31/496; A61K 31/495; A61K 31/4545; A61K 31/4439

(52) U.S. Cl. 514/183; 514/217.04; 514/227.8; 514/235.2; 514/253.01; 514/317; 514/318; 514/210.2; 514/341; 540/597; 544/59; 544/60; 544/124; 544/360; 546/193; 546/268.1; 548/950; 548/954

(57) ABSTRACT

Compounds of formula (I)

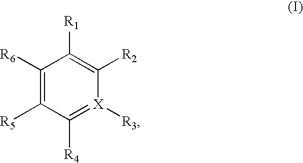

are useful for increasing cGMP levels in a mammal.

### TECHNICAL FIELD

The present invention relates to novel amides which are activators of soluble guanylate cyclase, their preparation and their use.

### BACKGROUND OF THE INVENTION

Soluble guanylate cyclase (sGC) catalyzes the conversion of guanosine 5′-triphosphate (GTP) to cyclic guanosine 3′,5′-monophosphate (cGMP). sGC is activated by nitric oxide (NO) binding to the enzyme. In response to stimulation by NO, soluble guanylate cyclase produces cGMP which serves as a second messenger for a variety of extracellular signals from neurotransmitters and hormones by way of its interaction with intracellular targets including kinases and ion channels.

Pharmacological stimulation of sGC resulting in increased cGMP levels opens a new approach for treatment or prevention of disorders associated with low levels of cGMP such as cardiovascular disease, atherosclerosis, angina pectoris, diastolic dysfunction, benign prostatic hyperplasia (BPH), incontinence, sexual dysfunction, endothelial dysfunction, trombosis, diabetes, liver cirhosis, and CNS disorders such as cognitive disorders, Alzheimer's disease, anxiety, stress, depression, sleep disorders, migraine, cerebral ischemia, brain trauma, pain, memory, and learning capabilities.

The present invention discloses novel amides that activate sGC resulting in increased levels of cGMP.

### SUMMARY OF THE INVENTION

The present invention discloses novel amide compounds, a method for activating soluble guanylate cyclase, and pharmaceutical compositions including those compounds. More particularly, the present invention is directed to compounds of formula (I)

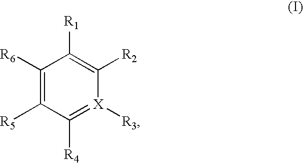

or a pharmaceutically acceptable salt, ester, amide or prodrug thereof, wherein

X is selected from C or N;

R₁ is selected from (NR7R8)carbonylalkyl-, or (NR7R8)carbonylalkenyl-;

R₂ is selected from alkoxy, alkylthio, aryloxy, arylthio, cycloalkyloxy, or cycloalkylthio;

provided that when X is C and R₂ is arylthio, then R₁ is (NR7R8)carbonylalkyl-;

R₃ is absent or selected from hydrogen, alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, or (NR9R10)carbonyl-;

R₄, R₅, and R₆ are independently selected from hydrogen, alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, or (NR9R10)carbonyl-;

R₇ and R₈ are independently selected from hydrogen, alkyl, aryl, arylalkyl, cycloalkyl, cycloalkylalkyl, heterocycle, heterocyclealkyl, hydroxyalkyl, or (NHR11)alkyl-; or

R₇ and R₈ taken together with the nitrogen atom to which they are attached, together form a heterocycle selected from 1-azetidinyl, 1-azepanyl, 1-aziridinyl, 4-morpholinyl, 1-piperazinyl, 1-piperidinyl, 1-pyrrolidinyl, and 1,1-dioxido-4-thiomorpholinyl, wherein the heterocycle is substituted with 0, 1, 2, 3, or 4 substituents selected from alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, or (NR9R10)carbonyl-;

R₉ and R₁₀ are independently selected from hydrogen or alkyl; and

R₁₁ is selected from hydrogen, alkoxy, alkyl, or alkylsulfonyl.

### DETAILED DESCRIPTION OF THE INVENTION

All patents, patent applications, and literature references cited in the specification are herein incorporated by reference in their entirety. In the case of inconsistencies, the present disclosure, including definitions, will prevail.

In its principle embodiment, the present invention relates to compounds of formula (I)

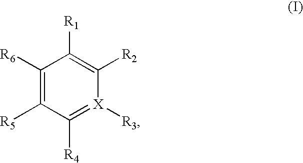

or a pharmaceutically acceptable salt, ester, amide or prodrug thereof, wherein

X is selected from C or N;

R₁ is selected from (NR7R8)carbonylalkyl-, or (NR7R8)carbonylalkenyl-;

R₂ is selected from alkoxy, alkylthio, aryloxy, arylthio, cycloalkyloxy, or cycloalkylthio;

provided that when X is C and R₂ is arylthio, then R₁ is (NR7R8)carbonylalkyl-;

R₃ is absent or selected from hydrogen, alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, or (NR9R10)carbonyl-;

R₄, R₅, and R₆ are independently selected from hydrogen, alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, or (NR9R10)carbonyl-;

R₇ and R₈ are independently selected from hydrogen, alkyl, aryl, arylalkyl, cycloalkyl, cycloalkylalkyl, heterocycle, heterocyclealkyl, hydroxyalkyl, or (NHR11)alkyl-; or

R₇ and R₈ taken together with the nitrogen atom to which they are attached, together form a heterocycle selected from 1-azetidinyl, 1-azepanyl, 1-aziridinyl, 4-morpholinyl, 1-piperazinyl, 1-piperidinyl, 1-pyrrolidinyl, and 1,1-dioxido-4-thiomorpholinyl, wherein the heterocycle is substituted with 0, 1, 2, 3, or 4 substituents selected from alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, or (NR9R10)carbonyl-;

R₉ and R₁₀ are independently selected from hydrogen or alkyl; and

R₁₁ is selected from hydrogen, alkoxy, alkyl, or alkylsulfonyl.

In another embodiment, the present invention relates to compounds of formula (I) wherein X is C; R₁ is (NR7R8)carbonylalkenyl-; R₂ is alkylthio; and R₃, R₄, R₅, R₆, R₇, and R₈ are as defined in formula (I).

In another embodiment, the present invention relates to compounds of formula (I) wherein X is C; R₁ is (NR7R8)carbonylalkenyl-; R₂ is alkylthio; R₃, R₄, R₅, and R₆ are independently selected from hydrogen, alkoxy, alkyl, or halogen; R₇ is cycloalkyl substituted with 0, 1, or 2 substituents selected from alkyl or hydroxy; and R₈ is selected from hydrogen or alkyl.

In another embodiment, the present invention relates to compounds of formula (I) wherein X is C; R₁ is (NR7R8)carbonylalkenyl-; R₂ is alkylthio; R₃, R₄, R₅, and R₆ are hydrogen; R₇ is cycloalkyl wherein the cycloalkyl is cyclohexyl substituted with hydroxy; and R₈ is hydrogen.

In another embodiment, the present invention relates to compounds of formula (I) wherein X is C; R₁ is (NR7R8)carbonylalkenyl-; R₂ is alkylthio wherein the alkylthio is 3-methylbutylthio; R₃, R₄, R₅, and R₆ are hydrogen; R₇ is cycloalkyl wherein the cycloalkyl is cyclohexyl substituted with hydroxy; and R₈ is hydrogen.

In another embodiment, the present invention relates to compounds of formula (I) wherein X is C; R₁ is (NR7R8)carbonylalkenyl-; R₂ is alkylthio wherein the alkylthio is 3-methylbutylthio; R₃, R₄, R₅, and R₆ are hydrogen; R₇ is cycloalkyl wherein the cycloalkyl is 4-hydroxycyclohexyl; and R₈ is hydrogen.

In another embodiment, the present invention relates to compounds of formula (I) wherein X is C; R₁ is (NR7R8)carbonylalkenyl- wherein the alkenyl of carbonylalkenyl is vinyl; R₂ is alkylthio wherein the alkylthio is 3-methylbutylthio; R₃, R₄, R₅, and R₆ are hydrogen; R₇ is cycloalkyl wherein the cycloalkyl is 4-hydroxycyclohexyl; and R₈ is hydrogen.

In another embodiment, the present invention relates to compounds of formula (I) wherein X is C; R₁ is (NR7R8)carbonylalkenyl-; R₂ is alkylthio; R₃, R₄, R₅, and R₆ are independently selected from hydrogen, alkoxy, alkyl, or halogen; R₇ is hydroxyalkyl; and R₈ is selected from hydrogen or alkyl.

In another embodiment, the present invention relates to compounds of formula (I) wherein X is C; R₁ is (NR7R8)carbonylalkenyl- wherein R₇ and R₈ taken together with the nitrogen atom to which they are attached, together form a heterocycle selected from 1-azetidinyl, 1-azepanyl, 1-aziridinyl, 4-morpholinyl, 1-piperidinyl, 1-pyrrolidinyl, and 1,1-dioxido-4-thiomorpholinyl, wherein the heterocycle is substituted with 0, 1, 2, 3, or 4 substituents selected from alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, or (NR9R10)carbonyl-; R₂ is alkylthio; and R₃, R₄, R₅, and R₆ are as defined in formula (I).

In another embodiment, the present invention relates to compounds of formula (I) wherein X is C; R₁ is (NR7R8)carbonylalkenyl- wherein R₇ and R₈ taken together with the nitrogen atom to which they are attached, together form 1-piperidinyl substituted with 0, 1, or 2 substituents selected from alkyl, hydroxy, hydroxyalkyl, or (NR9R10)carbonyl-; R₂ is alkylthio; and R₃, R₄, R₅, and R₆ are independently selected from hydrogen, alkoxy, alkyl, or halogen; and R₉ and R₁₀ are as defined in formula (I).

In another embodiment, the present invention relates to compounds of formula (I) wherein X is C; R₁ is (NR7R8)carbonylalkenyl-; R₂ is cycloalkylthio; and R₃, R₄, R₅, R₆, R₇, and R₈ are as defined in formula (I).

In another embodiment, the present invention relates to compounds of formula (I) wherein X is C; R₁ is (NR7R8)carbonylalkenyl-; R₂ is cycloalkylthio; R₃, R₄, R₅, and R₆ are independently selected from hydrogen, alkoxy, alkyl, or halogen; R₇ is cycloalkyl substituted with 0, 1, or 2 substituents selected from alkyl or hydroxy; and R₈ is selected from hydrogen or alkyl.

In another embodiment, the present invention relates to compounds of formula (I) wherein X is C; R₁ is (NR7R8)carbonylalkyl-; R₂ is arylthio; and R₃, R₄, R₅, R₆, R₇, and R₈ are as defined in formula (I).

In another embodiment, the present invention relates to compounds of formula (I) wherein X is C; R₁ is (NR7R8)carbonylalkyl-; R₂ is arylthio wherein the aryl of arylthio is phenyl substituted with 0, 1, or 2 substituents selected from alkoxy, alkyl, or halogen; R₃, R₄, R₅, and R₆ are independently selected from hydrogen, alkoxy, alkyl, or halogen; R₇ is cycloalkyl substituted with 0, 1, or 2 substituents selected from alkyl or hydroxy; and R₈ is selected from hydrogen or alkyl.

In another embodiment, the present invention relates to compounds of formula (I) wherein X is C; R₁ is (NR7R8)carbonylalkyl-; R₂ is arylthio wherein the aryl of arylthio is phenyl substituted with 0, 1, or 2 substituents selected from alkoxy, alkyl, or halogen; R₃, R₄, R₅, and R₆ are independently selected from hydrogen, alkoxy, alkyl, or halogen; R₇ is selected from hydroxyalkyl or (NHR11)alkyl-; R₈ is selected from hydrogen or alkyl; and R₁₁ is selected from hydrogen, alkoxy, alkyl, or alkylsulfonyl.

In another embodiment, the present invention relates to compounds of formula (I) wherein X is C; R₁ is (NR7R8)carbonylalkyl-; R₂ is arylthio wherein the aryl of arylthio is phenyl substituted with 0, 1, or 2 substituents selected from alkoxy, alkyl, or halogen; R₃, R₄, R₅, and R₆ are independently selected from hydrogen, alkoxy, alkyl, or halogen; R₇ is heterocycle; and R₈ is selected from hydrogen or alkyl.

In another embodiment, the present invention relates to compounds of formula (I) wherein X is C; R₁ is (NR7R8)carbonylalkyl-; R₂ is arylthio wherein the aryl of arylthio is substituted with 0, 1, or 2 substituents selected from alkoxy, alkyl, or halogen; R₃, R₄, R₅, and R₆ are independently selected from hydrogen, alkoxy, alkyl, or halogen; R₇ is heterocycle wherein the heterocycle is piperidinyl substituted with 0, 1, or 2 substituents selected from alkoxycarbonyl or alkyl; and R₈ is selected from hydrogen or alkyl.

In another embodiment, the present invention relates to compounds of formula (I) wherein X is C; R₁ is (NR7R8)carbonylalkyl-; R₂ is arylthio wherein the aryl of arylthio is phenyl substituted with 0, 1, or 2 substituents selected from alkoxy, alkyl or halogen; R₃, R₄, R₅, and R₆ are independently selected from hydrogen, alkoxy, alkyl, or halogen; R₇ is heterocyclealkyl; and R₈ is selected from hydrogen or alkyl.

In another embodiment, the present invention relates to compounds of formula (I) wherein X is C; R₁ is (NR7R8)carbonylalkyl-; R₂ is arylthio wherein the aryl of arylthio is phenyl substituted with 0, 1, or 2 substituents selected from alkoxy, alkyl, or halogen; R₃, R₄, R₅, and R₆ are independently selected from hydrogen, alkoxy, alkyl, or halogen; R₇ is heterocyclealkyl wherein the heterocycle of heterocyclealkyl is pyridinyl substituted with 0, 1, or 2 substituents selected from alkoxy, alkyl, or halogen; and R₈ is selected from hydrogen or alkyl.

In another embodiment, the present invention relates to compounds of formula (I) wherein X is N; R₁ is (NR7R8)carbonylalkenyl-; R₂ is arylthio; and R₃, R₄, R₅, R₆, R₇, and R₈ are as defined in formula (I).

In another embodiment, the present invention relates to compounds of formula (I) wherein X is N; R₁ is (NR7R8)carbonylalkenyl-; R₂ is arylthio wherein the aryl of arylthio is phenyl substituted with 0, 1, or 2 substituents selected from alkoxy, alkyl or halogen; R₃ is absent; R₄, R₅, and R₆ are independently selected from hydrogen, alkoxy, alkyl, or halogen; R₇ is cycloalkyl substituted with 0, 1, or 2 substituents selected from alkyl or hydroxy; and R₈ is selected from hydrogen or alkyl.

In another embodiment, the present invention relates to a method of treating a disorder ameliorated by increasing cGMP levels in a mammal comprising administering to the mammal a therapeutically effective amount of a compound of formula (I).

In another embodiment, the present invention relates to a method of treating cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory or learning disorders in a mammal comprising administering to the mammal a therapeutically effective amount of a compound of formula (I).

In another embodiment, the present invention relates to a method of treating sexual dysfunction in a mammal comprising administering to the mammal a therapeutically effective amount of a compound of formula (I).

In another embodiment, the present invention relates to a method of treating male erectile dysfunction in a mammal comprising administering to the mammal a therapeutically effective amount of a compound of formula (I).

In another embodiment, the present invention relates to a method of treating a disorder ameliorated by increasing cGMP levels in a mammal comprising administering to the mammal a therapeutically effective amount of a compound of formula (I) in combination with a pharmaceutically acceptable carrier.

In another embodiment, the present invention relates to a method of treating cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory or learning disorders in a mammal comprising administering to the mammal a therapeutically effective amount of a compound of formula (I) in combination with a pharmaceutically acceptable carrier.

In another embodiment, the present invention relates to a method of treating sexual dysfunction in a mammal comprising administering to the mammal a therapeutically effective amount of a compound of formula (I) in combination with a pharmaceutically acceptable carrier.

In another embodiment, the present invention relates to a method of treating male erectile dysfunction in a mammal comprising administering to the mammal a therapeutically effective amount of a compound of formula (I) in combination with a pharmaceutically acceptable carrier.

In another embodiment, the present invention relates to a method of treating a disorder ameliorated by increasing cGMP levels in a mammal comprising administering to the mammal a therapeutically effective amount of a compound of formula (I) in combination with a phosphodiesterase 5 inhibitor.

In another embodiment, the present invention relates to a method of treating cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory, or learning disorders in a mammal comprising administering to the mammal a therapeutically effective amount of a compound of formula (I) in combination with a phosphodiesterase 5 inhibitor.

In another embodiment, the present invention relates to a method of treating sexual dysfunction in a mammal comprising administering to the mammal a therapeutically effective amount of a compound of formula (I) in combination with a phosphodiesterase 5 inhibitor.

In another embodiment, the present invention relates to a method of treating male erectile dysfunction in a mammal comprising administering to the mammal a therapeutically effective amount of a compound of formula (I) in combination with a phosphodiesterase 5 inhibitor.

In another embodiment, the present invention relates to a method of treating a disorder ameliorated by increasing cGMP levels in a mammal comprising administering to the mammal a therapeutically effective amount of a compound of formula (I) in combination with an adrenergic receptor antagonist.

In another embodiment, the present invention relates to a method of treating cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory, or learning disorders in a mammal comprising administering to the mammal a therapeutically effective amount of a compound of formula (I) in combination with an adrenergic receptor antagonist.

In another embodiment, the present invention relates to a method of treating sexual dysfunction in a mammal comprising administering to the mammal a therapeutically effective amount of a compound of formula (I) in combination with an adrenergic receptor antagonist.

In another embodiment, the present invention relates to a method of treating male erectile dysfunction in a mammal comprising administering to the mammal a therapeutically effective amount of a compound of formula (I) in combination with an adrenergic receptor antagonist.

In another embodiment, the present invention relates to a method of treating a disorder ameliorated by increasing cGMP levels in a mammal comprising administering to the mammal a therapeutically effective amount of a compound of formula (I) in combination with a dopamine receptor agonist.

In another embodiment, the present invention relates to a method of treating cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory, or learning disorders in a mammal comprising administering to the mammal a therapeutically effective amount of a compound of formula (I) in combination with a dopamine receptor agonist.

In another embodiment, the present invention relates to a method of treating sexual dysfunction in a mammal comprising administering to the mammal a therapeutically effective amount of a compound of formula (I) in combination with a dopamine receptor agonist.

In another embodiment, the present invention relates to a method of treating male erectile dysfunction in a mammal comprising administering to the mammal a therapeutically effective amount of a compound of formula (I) in combination with a dopamine receptor agonist.

In another embodiment, the present invention relates to a method of treating sexual dysfunction in a mammal comprising administering to said mammal in need of such treatment a therapeutically effective amount of a compound of formula (II)

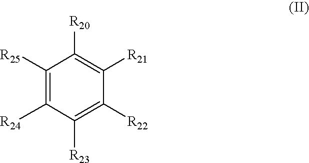

or a pharmaceutically acceptable salt, ester, amide, or prodrug thereof, wherein

R₂₀ is (NR₂₆R₂₇)carbonylalkenyl;

R₂₁ is arylthio wherein the aryl of arylthio is phenyl substituted with 0, 1, 2, 3, 4, or 5 substituents selected from alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₂₈R₂₉, or (NR₂₈R₂₉)carbonyl;

R₂₂, R₂₃, R₂₄, and R₂₅ are independently selected from hydrogen, alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₂₈R₂₉, or (NR₂₈R₂₉)carbonyl;

R₂₆ and R₂₇ are independently selected from hydrogen, alkyl, cycloalkyl, or hydroxyalkyl; and

R₂₈ and R₂₉ are independently selected from hydrogen or alkyl.

In another embodiment, the present invention relates to a method of treating sexual dysfunction in a mammal comprising administering to said mammal in need of such treatment a therapeutically effective amount of a compound of formula (II) wherein R₂₀ is (NR₂₆R₂₇)carbonylalkenyl; R₂₁ is arylthio wherein the aryl of arylthio is phenyl substituted with 0, 1, 2, or 3 substituents selected from alkoxy, alkyl, or halogen; R₂₂, R₂₃, R₂₄, and R₂₅ are independently selected from hydrogen, alkoxy, or halogen; R₂₆ is cycloalkyl substituted with 0, 1, or 2 substituents selected from alkyl, hydroxy, or oxo; and R₂₇ is selected from hydrogen or alkyl.

In another embodiment, the present invention relates to a method of treating sexual dysfunction in a mammal comprising administering to said mammal in need of such treatment a therapeutically effective amount of a compound of formula (II) wherein R₂₀ is (NR₂₆R₂₇)carbonylalkenyl; R₂₁ is arylthio wherein the aryl of arylthio is phenyl substituted with 0, 1, 2, or 3 subtituents selected from alkoxy, alkyl, or halogen; R₂₂, R₂₃, R₂₄, and R₂₅ are independently selected from hydrogen, alkoxy, or halogen; R₂₆ is selected from alkyl or hydroxyalkyl; R₂₇ is selected from hydrogen or alkyl.

### Definitions of the Present Invention

As used throughout this specification and the appended claims, the following terms have the following meanings:

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl (vinyl), 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy,” as used herein, refers to an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl,” as used herein, refers to an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxysulfonyl,” as used herein, refers to an alkoxy group, as defined herein, appended appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl,” as used herein, refers to an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy,” as used herein, refers to an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylsulfinyl,” as used herein, refers to an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfonyl,” as used herein, refers to an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio,” as used herein, refers to an alkyl group, as defined herein, appended to the parent molecular moiety through a sulfur atom. Representative examples of alkylthio include, but are not limited, methylthio, 3-methylbutylthio, ethylthio, tert-butylthio, and hexylthio.

The term “alkylthioalkyl,” as used herein, refers to an alkylthio group, as defined herein, appended to the parent molecular moiety through an alkyl group, as defined herein. Representative examples of alkylthioalkyl include, but are not limited, methylthiomethyl and 2-(ethylthio)ethyl.

The term “alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, refers to a phenyl group, or a bicyclic or a tricyclic fused ring system wherein one or more of the fused rings is a phenyl group. Bicyclic fused ring systems are exemplified by a phenyl group fused to a cycloalkyl group, as defined herein, or another phenyl group. Tricyclic fused ring systems are exemplified by a bicyclic fused ring system, as defined herein, fused to a cycloalkyl group, as defined herein, or another phenyl group. Representative examples of aryl include, but are not limited to, anthracenyl, azulenyl, fluorenyl, 2,3-dihydroindenyl, indenyl, naphthyl, phenyl, and tetrahydronaphthyl.

The aryl groups of this invention are substituted with 0, 1, 2, 3, 4 or 5 substituents independently selected from alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfonyl, alkylthio, alkylthioalkyl, alkynyl, carboxy, carboxyalkyl, cycloalkyl, cycloalkylalkyl, cyano, cyanoalkyl, ethylenedioxy, formyl, haloalkoxy, haloalkyl, halogen, hydroxy, hydroxyalkyl, mercapto, methylenedioxy, nitro, —NR_{A}R_{B}, (NR_{A}R_{B})carbonyl and (NR_{A}R_{B})sulfonyl. Representative examples of aryl substituted with 0, 1, 2, 3, 4 or 5 substituents include, but are not limited to, 4-methylphenyl, 4-bromophenyl, 4-chlorophenyl, 4-fluorophenyl, 4-methoxyphenyl, 2,4-dichlorophenyl, 2,4-dimethoxyphenyl, 2,4-dimethylphenyl, 2-methylphenyl, 2-methoxyphenyl, 2-bromophenyl, 2-chlorophenyl, 2-fluorophenyl, 3-methylphenyl, 3-methoxyphenyl, 3-bromophenyl, 3-chlorophenyl, and 3-fluorophenyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “aryloxy” as used herein, means an aryl group, as defined herein, appended to the parent molecular moiety through an oxygen atom. Representative examples of aryloxy include, but are not limited to, phenoxy, naphthyloxy, 4-methylphenoxy, 4bromophenoxy, 4-chlorophenoxy, 4-fluorophenoxy, 4-methoxyphenoxy, 2,4-dichlorophenoxy, 2,4-dimethoxyphenoxy, 2,4-dimethylphenoxy, 2-methylphenoxy, 2-methoxyphenoxy, 2-bromophenoxy, 2-chlorophenoxy, 2-fluorophenoxy, 3-methylphenoxy, 3-methoxyphenoxy, 3-bromophenoxy, 3-chlorophenoxy, and 3-fluorophenoxy.

The term “arylthio” as used herein, means an aryl group, as defined herein, appended to the parent molecular moiety through a sulfur atom. Representative examples of arylthio include, but are not limited to, phenylthio, 2-naphthylthio, 4-methylphenylthio, 4-bromophenylthio, 4-chlorophenylthio, 4-fluorophenylthio, 4-methoxyphenylthio, 2,4-dichlorophenylthio, 2,4-dimethoxyphenylthio, 2,4-dimethylphenylthio, 2-methylphenylthio, 2-methoxyphenylthio, 2-bromophenylthio, 2-chlorophenylthio, 2-fluorophenylthio, 3-methylphenylthio, 3-methoxyphenylthio, 3-bromophenylthio, 3-chlorophenylthio, and 3-fluorophenylthio.

The term “carbonyl,” as used herein, refers to a —C(O)— group.

The term “carboxy,” as used herein, refers to a —CO₂H group.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano,” as used herein, refers to a —CN group.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkyl” as used herein, means a saturated cyclic hydrocarbon group containing from 3 to 8 carbons. Examples of cycloalkyl include cyclopropyl, cyclobutyl, cyclopentyl, cyclohexyl, cycloheptyl, and cyclooctyl.

The cycloalkyl groups of this invention are substituted with 0, 1, 2, 3, or 4 substituents independently selected from alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfonyl, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, oxo, —NR_{A}R_{B}, (NR_{A}R_{B})carbonyl, and (NR_{A}R_{B})sulfonyl. Representative examples of cycloalkyl substituted with 0, 1, 2, 3, or 4 substituents include, but are not limited to, 4-hydroxycyclohexyl and 2-hydroxycyclohexyl.

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “cycloalkyloxy” as used herein, means cycloalkyl group, as defined herein, appended to the parent molecular moiety through an oxygen atom, as defined herein. Representative examples of cycloalkyloxy include, but are not limited to, cyclopropyloxy, cyclobutyloxy, cyclopentyloxy, cyclohexyloxy, cycloheptyloxy, and cyclooctyloxy.

The term “cycloalkylthio” as used herein, means cycloalkyl group, as defined herein, appended to the parent molecular moiety through a sulfur atom, as defined herein. Representative examples of cycloalkylthio include, but are not limited to, cyclopropylthio, cyclobutylthio, cyclopentylthio, cyclohexylthio, cycloheptylthio, and cyclooctylthio.

The term “ethylenedioxy,” as used herein, refers to a —O(CH₂)₂)— group wherein the oxygen atoms of the ethylenedioxy group are attached to the parent molecular moiety through one carbon atom forming a 5 membered ring or the oxygen atoms of the ethylenedioxy group are attached to the parent molecular moiety through two adjacent carbon atoms forming a six membered ring.

The term “formyl,” as used herein, refers to a —C(O)H group.

The term “halo” or “halogen,” as used herein, refers to —Cl, —Br, —I or —F.

The term “haloalkoxy,” as used herein, refers to at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic,” as used herein, refers to a monocyclic, bicyclic, or tricyclic ring system. Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered ring have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic ring systems fused to an aryl group as defined herein, a cycloalkyl group as defined herein, or another monocyclic ring system. Representative examples of bicyclic ring systems include but are not limited to, for example, benzimidazolyl, benzodioxinyl, benzothiazolyl, benzothienyl, benzotriazolyl, benzoxazolyl, benzofuranyl, benzopyranyl, benzothiopyranyl, cinnolinyl, indazolyl, indolyl, 2,3-dihydroindolyl, indolizinyl, naphthyridinyl, isobenzofuranyl, isobenzothienyl, isoindolyl, isoquinolinyl, phthalazinyl, pyranopyridinyl, quinolinyl, quinolizinyl, quinoxalinyl, quinazolinyl, tetrahydroisoquinolinyl, tetrahydroquinolinyl, and thiopyranopyridinyl. Tricyclic rings systems are exemplified by any of the above bicyclic ring systems fused to an aryl group as defined herein, a cycloalkyl group as defined herein, or a monocyclic ring system. Representative examples of tricyclic ring systems include, but are not limited to, acridinyl, carbazolyl, carbolinyl, dibenzo[b,d]furanyl, dibenzo[b,d]thienyl, naphtho[2,3-b]furan, naphtho[2,3-b]thienyl, phenazinyl, phenothiazinyl, phenoxazinyl, thianthrenyl, thioxanthenyl and xanthenyl.

The heterocycles of this invention are substituted with 0, 1, 2, or 3 substituents independently selected from alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfonyl, alkylthio, alkylthioalkyl, alkynyl, carboxy, carboxyalkyl, cyano, cyanoalkyl, ethylenedioxy, formyl, haloalkoxy, haloalkyl, halogen, hydroxy, hydroxyalkyl, mercapto, methylenedioxy, nitro, oxo, —NR_{A}R_{B}, (NR_{A}R_{B})carbonyl and (NR_{A}R_{B})sulfonyl.

Representative examples of heterocycle substituted with 0, 1, 2, 3, or 4 substituents include, but are not limited to, 4-hydroxy-1-piperidinyl, 1-methyl-4-piperidinyl, 3-hydroxy-1-piperidinyl, 4-(2-hydroxyethyl)-1-piperidinyl, 4-(aminocarbonyl)-1-piperidinyl.

The term “heterocyclealkenyl” as used herein, means a heterocycle, as defined herein, appended to the parent molecular moiety through an alkenyl group, as defined herein. Representative examples of heterocyclealkenyl include, but are not limited to, 3-pyridin-3-yl-2-propenyl, and 4-pyrimidin-2-yl-3-butenyl.

The term “heterocyclealkyl” as used herein, means a heterocycle, as defined herein, appended to the parent molecular moiety through an alkyl group, as defined herein. Representative examples of heterocyclealkyl include, but are not limited to, 2-(4-pyridinyl)ethyl, 3-pyridinylmethyl, 3-(2-pyrimidinyl)propyl, 2-(4-morpholinyl)ethyl, and 3-(1H-imidazol-1-yl)propyl.

The term “heterocyclecarbonyl” as used herein, means a heterocycle selected from aziridinyl, azetidinyl, as defined herein, appended to the parent molecular moiety through a carbonyl group, as defined herein. Representative examples of heterocyclecarbonyl include, but are not limited to, 3-pyridinylcarbonyl, 4-morpholinylcarbonyl, 1-piperazinylcarbonyl, and {4-[3-(dimethylamino)propyl]-1-piperazinyl}carbonyl.

The term “hydroxy,” as used herein, refers to an —OH group.

The term “hydroxyalkyl,” as used herein, refers to at least one hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2-ethyl-4-hydroxyheptyl and 2,3-dihydroxypropyl.

The term “mercapto,” as used herein, refers to a —SH group.

The term “mercaptoalkyl” as used herein, means a mercapto group, as defined herein, appended to the parent molecular moiety through an alkyl group, as defined herein. Representative examples of mercaptoalkyl include, but are not limited to, 2-mercaptoethyl and 3-mercaptopropyl.

The term “methylenedioxy,” as used herein, refers to a —OCH₂O— group wherein the oxygen atoms of the methylenedioxy are attached to the parent molecular moiety through two adjacent carbon atoms.

The term “nitro,” as used herein, refers to a —NO₂ group.

The term “—NR_{A}R_{B},” as used herein, refers to two groups, R_{A} and R_{B}, which are appended to the parent molecular moiety through a nitrogen atom. R_{A} and R_{B} are each independently selected from hydrogen, alkyl, alkylcarbonyl, or formyl. Representative examples of —NR_{A}R_{B} include, but are not limited to, amino, methylamino, dimethylamino, acetylamino, and ethylmethylamino.

The term “(NR_{A}R_{B})carbonyl,” as used herein, refers to a —NR_{A}R_{B} group, as defined herein, appended to the parent molecular moiety through a carbonyl group, as defined herein. Representative examples of (NR_{A}R_{B})carbonyl include, but are not limited to, aminocarbonyl, (methylamino)carbonyl, (dimethylamino)carbonyl, and (ethylmethylamino)carbonyl.

The term “(NR_{A}R_{B})sulfonyl,” as used herein, refers to a —NR_{A}R_{B} group, as defined herein, appended to the parent molecular moiety through a sulfonyl group, as defined herein. Representative examples of (NR_{A}R_{B})sulfonyl include, but are not limited to, aminosulfonyl, (methylamino)sulfonyl, (dimethylamino)sulfonyl, and (ethylmethylamino)sulfonyl.

The term “oxo,” as used herein, refers to a ═O moiety.

The term “sulfinyl,” as used herein, refers to a —S(O)— group.

The term “sulfonyl,” as used herein, refers to a —SO₂— group.

The term “sexual dysfunction,” as used herein refers to male sexual dysfunction and female sexual dysfunction.

The term “male sexual dysfunction,” as used herein includes, but is not limited to, male erectile dysfunction and premature ejacualtion.

The term “female sexual dysfunction,” as used herein includes, but is not limited to, female anorgasmia, clitoral erectile insufficiency, vaginal engorgement, dyspareunia, and vaginismus.

Compounds of the present invention may exist as stereoisomers wherein, asymmetric or chiral centers are present. These stereolsomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. The present invention contemplates various stereoisomers and mixtures thereof and are specifically included within the scope of this invention. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Compounds of the present invention that contain (NR7R8)carbonylalkenyl- are either cis or trans about the double bond or are a mixture of cis and trans. The isomers are designated as (Z) or (E) in accordance with ACD/ChemSketch version 5.0.

Compounds of the present invention were named by ACD/ChemSketch version 5.0 (developed by Advanced Chemistry Development, Inc., Toronto, ON, Canada) or were given names which appeared to be consistent with ACD nomenclature.

Representative compounds of the present invention include:

3-{2-[(4-chlorophenyl)thio]phenyl}-N-(4-hydroxybutyl)propanamide;

3-{2-[(4-chlorophenyl)thio]phenyl}-N-(5-hydroxy-1,5-dimethylhexyl)propanamide;

3-{2-[(4-chlorophenyl)thio]phenyl}-N-(5-hydroxypentyl)propanamide;

3-{2-[(4-chlorophenyl)thio]phenyl}-N-(4-hydroxycyclohexyl)propanamide;

N-(4-hydroxybutyl)-3-{2-[(4-methylphenyl)thio]phenyl}propanamide;

3-{2-[(4-chlorophenyl)thio]phenyl}-N-{4-[(methylsulfonyl)amino]butyl}propanamide;

3-{2-[(4-chlorophenyl)thio]-3-fluorophenyl}-N-(4-hydroxybutyl)propanamide;

3-{2-[(4-chlorophenyl)thio]-5-fluorophenyl}-N-(4-hydroxybutyl)propanamide;

3-{2-[(4-chlorophenyl)thio]phenyl}-N-(4-hydroxypentyl)propanamide;

3-{2-[(4-chlorophenyl)thio]phenyl}-N-methyl-N-(1-methyl-4-piperidinyl)butanamide;

3-{2-[(4-chlorophenyl)thio]phenyl}-N-[2-(4-pyridinyl)ethyl]butanamide;

3-{2-[(4-chlorophenyl)thio]phenyl}-N-[4-(methoxyamino)butyl]propanamide;

3-{2-[(4-chlorophenyl)thio]phenyl}-N-[4-(methylamino)butyl]propanamide;

3-{2-[(4-chlorophenyl)thio]phenyl}-N-[5-(methylamino)pentyl]propanamide;

(2E)-N-(4-hydroxycyclohexyl)-3-{2-[(4-methylphenyl)thio]-3-pyridinyl}acrylamide;

(2E)-3-{2-[(4-chlorophenyl)thio]-3-pyridinyl}-N-(4-hydroxycyclohexyl)acrylamide;

(2E)-3-{2-[(2,4-dichlorophenyl)thio]-3-pyridinyl}-N-(4-hydroxycyclohexyl)acrylamide;

(2E)-3-{2-[(4-bromophenyl)thio]-3-pyridinyl}-N-(4-hydroxycyclohexyl)acrylamide;

(2E)-N-(4-hydroxycyclohexyl)-3-[2-(phenylthio)-3-pyridinyl]acrylamide;

(2E)-3-{2-[(2-chlorophenyl)thio]-3-pyridinyl}-N-(4-hydroxycyclohexyl)acrylamide;

(2E)-N-(2-hydroxycyclohexyl)-3-[2-(isobutylthio)phenyl]acrylamide;

(2E)-3-[2-(cyclopentylthio)phenyl]-N-(2-hydroxycyclohexyl)acrylamide;

(2E)-3-[2-(cyclohexylthio)phenyl]-N-(2-hydroxycyclohexyl)acrylamide;

(2E)-N-(4-hydroxycyclohexyl)-3-[2-(isopropylthio)phenyl]acrylamide;

(2E)-3-[2-(cyclopentylthio)phenyl]-N-(4-hydroxycyclohexyl)acrylamide;

(2E)-3-[2-(cyclohexylthio)phenyl]-N-(4-hydroxycyclohexyl)acrylamide;

(2E)-N-(4-hydroxycyclohexyl)-3-[2-(pentylthio)phenyl]acrylamide;

(2E)-3-{2-[(4-chlorophenyl)thio]-6-methyl-3-pyridinyl}-N-(4-hydroxycyclohexyl)acrylamide;

(2E)-3-{2-[(2,4-dichlorophenyl)thio]phenyl}-N-(4-hydroxybutyl)acrylamide;

(2E)-3-{2-[(4-chlorophenyl)thio]phenyl}-N-(4-hydroxycyclohexyl)acrylamide;

(2E)-3-{2-[(4-chlorophenyl)thio]phenyl}-N-(5-hydroxy-1,5-dimethylhexyl)acrylamide;

(2E)-3-{2-[(4-chlorophenyl)thio]phenyl}-N-ethylacrylamide;

(2E)-N-butyl-3-{2-[(4-chlorophenyl)thio]phenyl}acrylamide;

(2E)-3-{2-[(4-chlorophenyl)thio]phenyl}-N-(4-hydroxybutyl)acrylamide;

(2E)-3-{2-[(4-chlorophenyl)thio]phenyl}-N-(5-hydroxypentyl)acrylamide;

(2E)-N-(4-hydroxybutyl)-3-{2-[(4-methylphenyl)thio]phenyl}acrylamide;

(2E)-3-{2-[(4-chlorophenyl)thio]phenyl}-N-(2-hydroxypropyl)acrylamide;

(2E)-3-{2-[(4-chlorophenyl)thio]phenyl}-N-(3-hydroxybutyl)acrylamide;

(2E)-3-{2-[(4-chlorophenyl)thio]phenyl}-N-(2-hydroxybutyl)acrylamide;

(2E)-3-{2-[(4-chlorophenyl)thio]phenyl}-N-(2-hydroxycyclohexyl)acrylamide;

(2E)-3-{2-[(4-chlorophenyl)thio]-6-fluorophenyl}-N-(4-hydroxybutyl)acrylamide;

(2E)-N-(4-hydroxycyclohexyl)-3-{2-[(4-methylphenyl)thio]phenyl}acrylamide;

(2E)-N-(4-hydroxybutyl)-3-{2-[(4-methoxyphenyl)thio]phenyl}acrylamide;

(2E)-N-(4-hydroxycyclohexyl)-3-{5-methoxy-2-[(4-methylphenyl)thio]phenyl}acrylamide;

(2E)-3-{2-[(4-fluorophenyl)thio]phenyl}-N-(2-hydroxycyclohexyl)acrylamide;

(2E)-N-(2-hydroxycyclohexyl)-3-[2-(phenylthio)phenyl]acrylamide;

(2E)-N-(2-hydroxycyclohexyl)-3-{2-[(4-methylphenyl)thio]phenyl}acrylamide;

(2E)-N-(2-hydroxycyclohexyl)-3-{2-[(3-methoxyphenyl)thio]phenyl}acrylamide;

(2E)-N-(2-hydroxycyclohexyl)-3-{2-[(4-methoxyphenyl)thio]phenyl}acrylamide;

(2E)-N-(4-hydroxycyclohexyl)-3-{2-[(3-methoxyphenyl)thio]phenyl}acrylamide;

(2E)-N-(4-hydroxycyclohexyl)-3-{2-[(4-methoxyphenyl)thio]phenyl}acrylamide;

(2E)-N-(4-hydroxycyclohexyl)-3-{2-[(3-methylphenyl)thio]phenyl}acrylamide;

(2E)-3-{2-[(2,4-dimethylphenyl)thio]phenyl}-N-(5-hydroxy-1,5-dimethylhexyl)acrylamide;

(2E)-3-{2-[(4-chlorophenyl)thio]-5-fluorophenyl}-N-(4-hydroxybutyl)acrylamide;

(2E)-N-(2-hydroxycyclohexyl)-3-{2-[(3-methylbutyl)thio]phenyl}acrylamide;

(2E)-N-(3-hydroxypropyl)-3-{2-[(3-methylbutyl)thio]phenyl}acrylamide;

(2E)-N-(4-hydroxybutyl)-3-{2-[(3-methylbutyl)thio]phenyl}acrylamide;

(2E)-N-(5-hydroxypentyl)-3-{2-[(3-methylbutyl)thio]phenyl}acrylamide;

1-((2E)-3-{2-[(3-methylbutyl)thio]phenyl}-2-propenoyl)-4-piperidinol;

1-((2E)-3-{2-[(3-methylbutyl)thio]phenyl}-2-propenoyl)-3-piperidinol;

2-[1-((2E)-3-{2-[(3-methylbutyl)thio]phenyl}-2-propenoyl)-4-piperidinyl]ethanol; and

1-((2E)-3-{2-[(3-methylbutyl)thio]phenyl}-2-propenoyl)-4-piperidinecarboxamide; or a pharmaceutically acceptable salt, ester, amide or prodrug thereof

A preferred compound of the present invention is (2E)-N-(4-hydroxycyclohexyl)-3-{2-[(3-methylbutyl)thio]phenyl}acrylamide.

### Abbreviations

Abbreviations which have been used in the descriptions of the Schemes and the Examples that follow are: DCC for 1,3-dicyclohexylcarbodiimide; DIBAL for diisobutylaluminum hydride; DMA for dimethylacetamide; DCM for dichloromethane; DIAD for diisopropyl azodicarboxylate; DMF for N,N-dimethylformamide; DMSO for dimethylsulfoxide; EDCI or EDC for 1-ethyl-3-[3-(dimethylamino)propyl]-carbodiimide hydrochloride; Et for ethyl; EtOAc for ethyl acetate; EtOH for ethanol; LDA for lithium diisopropylamine; MeOH for methanol; Ms for CH₃S(O)₂; PCC for pyridinium chlorochromate; TEA for triethylamine; THF for tetrahydrofuran; and Ts for p-CH₃C₆H₅S(O)₂.

### Preparation of Compounds of the Present Invention

The compounds of the present invention may be prepared by a variety of synthetic routes. Representative procedures are described in Schemes 1-4.

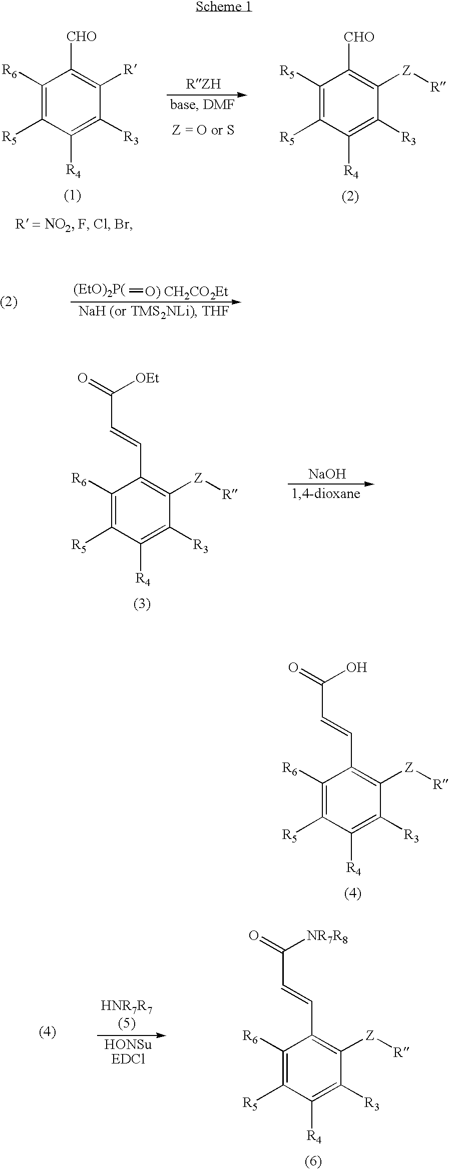

Amides of general formula (6), wherein R₃, R₄, R₅, R₆, R₇, and R₈ are as defined in formula (I), Z is O or S, and R″ is alkyl, aryl, or cycloalkyl, can be prepared as described in Scheme 1. Aldehydes of general formula (I), purchased commercially or prepared using standard methods known to those of ordinary skill in the art, can be treated with alcohols, phenols, cycloalkylhydroxy compounds, alkylthiols, cycloalkylthiols, or arylthiols and a base, including but not limited to, potassium carbonate, sodium bicarbonate, triethylamine, or diisopropylethylamine to provide aldehydes of general formula (2). Aldehydes of general formula (2) can be treated with triethylphosphonoacetate and a base such as sodium hydride or lithium bis(trimethylsilyl)amide to provide enoates of general formula (3). Enoates of general formula (3) can be treated with aqueous bases such as sodium hydroxide in a solvent miscible with water such as THF or 1,4-dioxane to provide acids of general formula (4). Acids of general formula (4) can be treated with amines of general formula (5), a carbodiimide such as EDCI or DCC, and N-hydroxysuccinimide (HONSu) to provide amides of general formula (6).

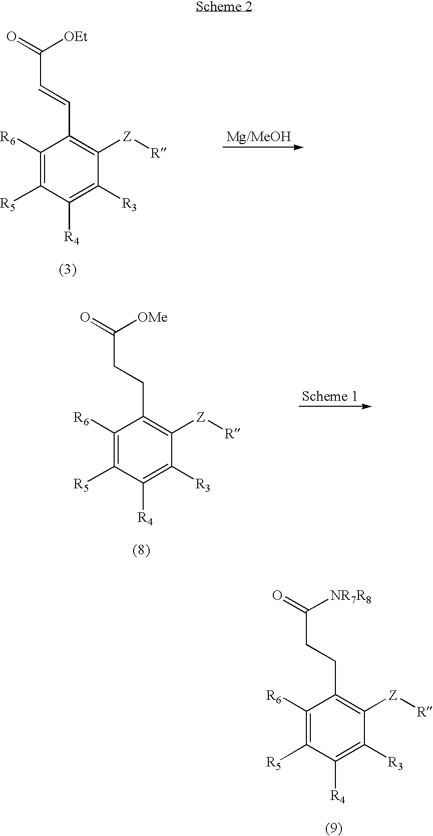

Amides of general formula (9), wherein R₃, R₄, R₅, R₆, R₇, and R₈ are as defined in formula (I), Z is O or S, and R″ is alkyl, aryl, or cycloalkyl, can be prepared as described in Scheme 2. Compounds of general formula (3) can be treated with magnesium turnings to provide esters of general formula (8). Esters of general formula (8) can be processed as described in Scheme 1 to provide amides of general formula (9).

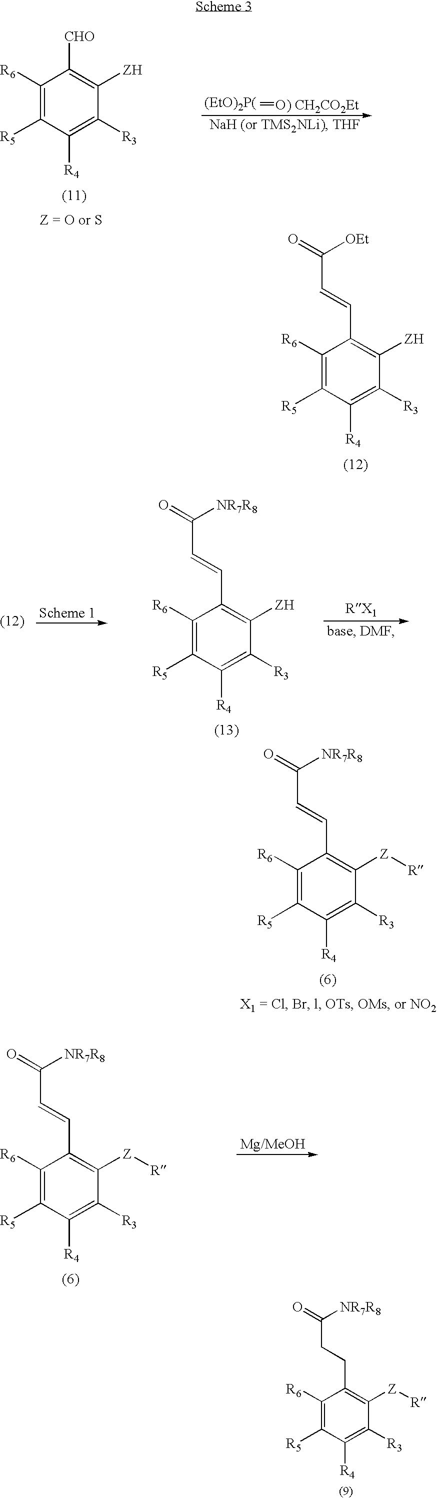

Amides of general formula (6) or amides of general formula (9), wherein R₃, R₄, R₅, R₆, R₇, and R₈ are as defined in formula (I), Z is O or S, and R″ is alkyl, aryl, or cycloalkyl, can be prepared as described in Scheme 3. Aldehydes of general formula (11), purchased commercially or prepared using standard methods known to those of ordinary skill in the art, can be treated with triethylphosphonoacetate and a base such as sodium hydride or lithium bis(trimethylsilyl)amide to provide enoates of general formula (12). Enoates of general formula (12) can be treated as described in Scheme 1 to provide amides of general formula (13). Amides of general formula (13) can be treated with alkyl halides, alkyl mesylates, alkyl tosylates, aryl halides, aryl mesylates, aryl tosylates, cycloalkyl halides, cycloalkyl mesylates, or cycloalkyl tosylates and a base to provide amides of general formula (6).

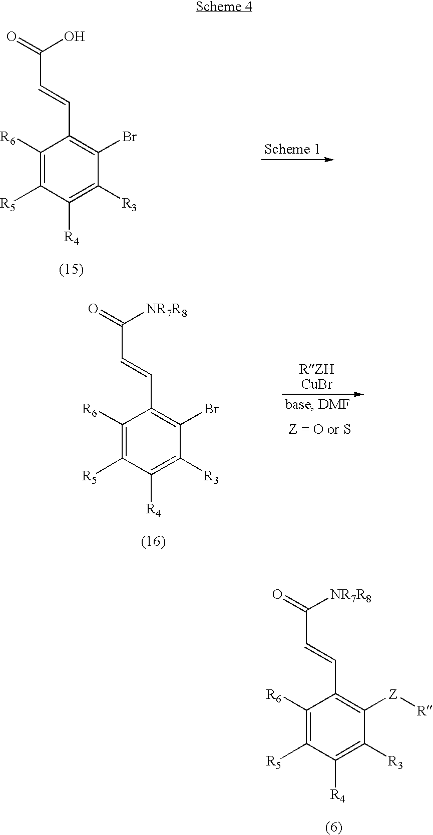

Amides of general formula (6), wherein R₃, R₄, R₅, R₆, R₇, and R₈ are as defined in formula (I), Z is O or S, and R″ is alkyl, aryl, or cycloalkyl, can be prepared as described in Scheme 4. 3-(2-Bromophenyl)acrylic acid, purchased from Aldrich, can be treated as described in Scheme 1 to provide amides of general formula (16). Amides of general formula (16) can be treated with CuBr, and alcohols, phenols, cycloalkylhydroxy compounds, alkylthiols, cycloalkylthiols, or arylthiols and a base to provide amides of general formula (6).

### EXAMPLE 1

### 3-{2-[(4-chlorophenyl)thio]phenyl}-N-(4-hydroxybutyl)propanamide

### EXAMPLE 1A

### ethyl 3-{2-[(4-chlorophenyl)thio]phenyl}acrylate

Triethyl phosphonoacetate (2.1 mL, 10 mmol) in THF (40 mL) at room temperature was treated with 60% oil dispersion NaH (0.4 g, 10 mmol) followed by addition of 2[(4-chlorophenyl)thio]benzaldehyde (2.49 g, 10 mmol). The mixture was stirred at room temperature for 3 hours and concentrated under reduced pressure. The residue was treated with 10% citric acid and extracted with ethyl acetate. The organic extract was washed with water, brine, dried with MgSO₄, filtered, and the filtrate was concentrated under reduced pressure to provide the title compound. ¹H NMR (300 MHz, CDCl₃) δ 1.30 (7, J=7 Hz, 3H), 4.24 (q, J=7 Hz, 2H), 6.37 (d, J=15 Hz, 1H), 7.16 (d, J=9 Hz, 1H), 7.23 (m, 2H), 7.42 (m, 3H), 7.63 (m, 1H), 8.21 (d, J=15 Hz, 1H); MS (DCI/NH₃) m/z 336 (M+NH₄)⁺.

### EXAMPLE 1B

### methyl 3-{2-[(4-chlorophenyl)thio]phenyl}propanoate

The product from Example 1A (1.6 g, 5 mmol) in methanol (50 mL) was treated with Mg turnings (1.2 g) and stirred for 8 hours at room temperature. The mixture was concentrated under reduced pressure, treated with 1N HCl, and extracted with ethyl acetate to provide the title compound. ¹H NMR (300 MHz, CDCl₃) δ 2.62 (t, J=7 Hz, 2H), 3.10 (t, J=7 Hz, 2H), 3.67 (s, 3H), 7.12 (d, J=9 Hz, 2H), 7.25 (m, 6H); MS (DCI/NH₃) m/z 324 (M+NH₄)⁺.

### EXAMPLE 1C

### 3-{2-[(4-chlorophenyl)thio]phenyl}propanoic acid

The product from Example 1B (1.5 g, 5 mmol) and 1N NaOH (8 mL) in methanol:1,4-dioxane (2:1, 30 mL) was refluxed at 80° C. for 2 hours. The mixture was allowed to cool to room temperature, water was added, and the organics were removed under reduced pressure. The water layer was acidified with 10% citric acid and extracted with ethyl acetate to provide the title compound. ¹H NMR (300 MHz, DMSO-d₆) δ 2.50 (t, J=7 Hz, 2H), 2.93 (t, J=7 Hz, 2H), 7.12 (d, J=9 Hz, 2H), 7.33 (m, 6H), 12.20 (br s, 1H); MS (DCI/NH₃) m/z 310 (M+NH₄)⁺.

### EXAMPLE 1D

### 3-{2-[(4-chlorophenyl)thio]phenyl}-N-(4-hydroxybutyl)propanamide

The product from Example 1C (438 mg, 1.5 mmol), N-hydroxysuccinimide (167 mg, 1.5 mmol) and 4-amino-1-butanol (180 mg, 2 mmol) were combined in CHCl₃ (40 mL) and treated with EDCI (384 mg, 2 mmol). The mixture was stirred at room temperature for 12 hours and then concentrated under reduced pressure. The residue was partitioned between ethyl acetate and water and the phases separated. The organic layer was washed with water, brine, dried with MgSO₄, filtered and the filtrate concentrated under reduced pressure. The residue was purified by column chromatography (silica gel, ethyl acetate) to provide the title compound. MS (DCI/NH₃) m/z 364 (M+H)⁺; FAB HRMS m/z for C₁₉H₂₂ClNO₂S (M+H)⁺: calcd 364.1138, found 364.1151.

### EXAMPLE 2

### 3-{2-[(4-chlorophenyl)thio]phenyl}-N-(5-hydroxy-1,5-dimethylhexyl)propanamide

The product from Example 1C and 6-amino-2-methyl-2-heptanol were processed as described in Example 1D to provide the title compound. ¹H NMR (300 MHz, DMSO-d₆) δ 0.97 (d, J=7 Hz, 3H), 1.35 (s, 6H), 1.28 (m, 6H), 2.35 (t, J=7 Hz, 2H), 2.92 (t, J=7 Hz, 2H), 3.72 (m, 1H), 4.04 (s, 1H), 7.16 (d, J=9 Hz, 2H), 7.30 (m, 6H), 7.60 (d, J=7 Hz, 1H); MS (DCI/NH₃) m/z 420 (M+H)⁺. Analysis calcd for C₂₃H₃₀ClNO₂S.0.4H₂O: C, 64.66; H, 7.27; N, 3.28. Found: C, 64.65; H, 7.32; N, 3.73.

### EXAMPLE 3

### 3-{2-[(4-chlorophenyl)thio]phenyl}-N-(5-hydroxypentyl)propanamide

The product from Example 1C and 5-amino-1-pentanol were processed as described in Example 1D to provide the title compound. ¹H NMR (300 MHz, CDCl₃) δ 1.22 (m, 2H), 1.36 (m, 4H), 2.35 (t, J=9 Hz, 2H), 2.93 (t, J=9 Hz, 2H), 3.00 (d, J=9 Hz, 2H), 3.37 (m, 2H), 4.33 (t, J=4.5 Hz, 1H), 7.15 (m, 2H), 7.28 (m, 4H), 7.39 (m, 2H), 7.78 (t, J=6 Hz, 1H); MS (DCI/NH₃) m/z 378 (M+H)⁺. FAB HRMS m/z for C₂₀CH₂₅ClNO₂S (M+H)⁺: calcd 378.1295, found 378.1288.

### EXAMPLE 4

### 3-{2-[(4-chlorophenyl)thio]phenyl}-N-(4-hydroxycyclohexyl)propanamide

The product from Example 1C and 4-aminocyclohexanol were processed as described in Example 1D to provide the title compound. ¹H NMR (300 MHz, CDCl₃) δ 1.18 (m, 4H), 1.78 (m, 4H), 2.35 (t, J=9 Hz, 2H), 2.93 (t, J=7 Hz, 2H), 3.47 (m, 2H), 4.49 (d, J=3 Hz, 1H), 7.14 (m, 2H), 7.28 (m, 4H), 7.38 (m, 2H), 7.63 (d, J=12 Hz, 1H); MS (DCI/NH₃) m/z 390 (M+H)⁺.

### EXAMPLE 5

### N-(4-hydroxybutyl)-3-{2-[(4-methylphenyl)thio]phenyl}propanamide

### EXAMPLE 5A

### 2-[(4-methylphenyl)thio]benzaldehyde

2-Nitrobenzaldehyde (3.02 g, 20 mmol), 4-methylthiophenol (3 g, 22 mmol) and K₂CO₃ (5.52 g, 40 mmol) were combined in DMF (100 mL) and stirred at 50° C. for 24 hours. The mixture was poured into water (300 mL) and extracted with ethyl acetate. The acetate layer was washed with water, brine, dried over anhydrous MgSO₄, filtered, and the filtrate concentrated under reduced pressure. The residue was purified by column chromatography to provide the title compound. ¹H NMR (300 MHz, DMSO-d₆) δ 2.36 (s, 3H), 6.90 (d, J=9 Hz, 1H), 7.39 (m, 5H), 7.50 (m, 1H), 7.94 (dd, J=3 Hz, 7 Hz, 1H), 10.22 (s, 1H); MS (DCI/NH₃) m/z 229 (M+H)⁺, 246 (M+NH₄)⁺.

### EXAMPLE 5B

### ethyl 3-{2-[(4-methylphenyl)thio]phenyl}acrylate

Triethyl phosphonoacetate and the product from Example 5A were processed as described in Example 1A to provide the title compound.

### EXAMPLE 5C

### methyl 3-{2-[(4-methylphenyl)thio]phenyl}propanoate

The product from Example 5B was processed as described in Example 1B to provide the title compound.

### EXAMPLE 5D

### 3-{2-[(4-methylphenyl)thio]phenyl}propanoic acid

The product from Example 5C was processed as described in Example 1C to provide the title compound.

### EXAMPLE 5E

### N-(4-hydroxybutyl)-3-{2-[(4-methylphenyl)thio]phenyl}propanamide

The product from Example 5D and 4-amino-1-butanol were processed as described in Example 1D to provide the title compound. ¹H NMR (300 MHz, CDCl₃) δ 1.52 (m, 4H), 2.32 (s, 3H), 2.45 (t, J=7 Hz, 2H), 3.11 (t, J=7 Hz, 2H), 3.25 (m, 2H), 3.63 (t, J=7 Hz, 2H), 7.14 (d, J=9 Hz, 2H), 7.33 (m, 6H); MS (DCI/NH₃) m/z 344 (M+H)⁺. FAB HRMS m/z for C₂₀H₂₆NO₂S (M+H)⁺: calcd 344.1684, found 344.1676.

### EXAMPLE 6

### 3-{2-[(4-chlorophenyl)thio]phenyl}-N-{4-[(methylsulfonyl)amino]butyl}propanamide

Methane sulfonamide (25 mg, 0.26 mmol) in N,N-dimethylformamide (2 mL) at 0° C. solution was treated with small portions of NaH (14 mg, 0.34 mmol, 60% dispersion in oil). After complete addition, the cooling bath was removed and the mixture was warmed to 23° C. for 30 minutes and treated with the product from Example 16A (116 mg, 0.26 mmol) in N,N-dimethylformamide (2 mL). The mixture was heated at 140° C. for 18 hours, allowed to cool to ambient temperature and quenched with saturated ammonium chloride followed by 2 N hydrochloric acid. The solution was extracted three times with ethyl acetate (50 mL). The combined ethyl acetate phases were washed with brine, dried over MgSO₄, filtered, and the filtrate concentrated under reduced pressure. The residue was purified by chromatography (silica gel, ethyl acetate) to provide the title compound. ¹H NMR (300 MHz, CDCl₃) δ 1.50 (m, 4H), 2.43 (t, J=7.5 Hz, 2H), 2.93 (s, 3H), 3.11 (m, 4H), 3.24 (m, 2H), 4.43 (m, 1H), 5.39 (m, 1H), 7.11 (m, 2H), 7.26 (m, 6H); MS (APCI+) m/z 441 (M+H)⁺; FAB HRMS m/z for C₂₀H₂₅ClN₂O₃S₂ (M+H)⁺: calcd 441.1073, found 441.1074.

### EXAMPLE 7

### 3-{2-[(4-chlorophenyl)thio]-3-fluorophenyl}-N-(4-hydroxybutyl)propanamide

### EXAMPLE 7A

### 2-[(4-chlorophenyl)thio]-3-fluorobenzaldehyde

2,3-Difluorobenzaldehyde (1 g, 7 mmol), 4-chlorothiophenol (1 g, 7 mmol) and K₂CO₃ (1.4 g, 10 mmol) were combined in DMF (20 mL) and stirred at 50° C. for 16 hours. The mixture was poured into water (100 mL) and extracted with ethyl acetate. The acetate layer was washed with water, brine, dried over anhydrous MgSO₄, filtered, and the filtrate was concentrated under reduced pressure. The residue was purified by column chromatography to provide the title compound. ¹H NMR (300 MHz, DMSO-d₆) δ 7.23 (d, J=9 Hz, 2H), 7.40 (d, J=9 Hz, 2H), 7.75 (m, 3H), 10.48 (s, 1H); MS (DCI/NH₃) m/z 267 (M+H)⁺, 284 (M+NH₄)⁺.

### EXAMPLE 7B

### ethyl (2E)-3-{2-[(4-chlorophenyl)thio]-3-fluorophenyl}acrylate

The product from Example 7A and triethyl phosphonoacetate were processed as described in Example 1A to provide the title compound.

### EXAMPLE 7C

### methyl 3-{2-[(4-chlorophenyl)thio]-3-fluorophenyl}propanoate

The product from Example 7B was processed as described in Example 1B to provide the title compound.

### EXAMPLE 7D

### 3-{2-[(4-chlorophenyl)thio]-3-fluorophenyl}propanoic acid

The product from Example 7C was processed as described in Example 1C to provide the title compound.

### EXAMPLE 7E

### 3-{2-[(4-chlorophenyl)thio]-3-fluorophenyl}-N-(4-hydroxybutyl)propanamide

The product from Example 7D and 4-amino-1-butanol were processed as described in Example 1D to provide the title compound. ¹H NMR (300 MHz, DMSO-d₆) δ 1.35 (m, 4H), 2.33 (t, J=9 Hz, 2H), 2.95 (t, J=9 Hz, 2H), 3.01 (d, J=9 Hz, 2H), 3.38 (d, J=9 Hz, 2H), 4.35 J=4.5 Hz, 1H), 7.04 (m, 2H), 7.26 (m, 2H), 7.33(m, 2H), 7.48(m, 1H), 7.76 (t, J=6 Hz, 1H); MS (DCI/NH₃) m/z 382 (M+H)⁺. Analysis calcd for C₁₉H₂₁ClFNO₂S: C, 59.76; H, 5.54; N, 3.67. Found: C, 59.64; H, 5.46; N, 3.63.

### EXAMPLE 8

### 3-{2-[(4-chlorophenyl)thio]-5-fluorophenyl}-N-(4-hydroxybutyl)propanamide

### EXAMPLE 8A

### 2-[(4-chlorophenyl)thio]-5-fluorobenzaldehyde

2,5-Difluorobenzaldehyde (2.3 g, 20 mmol), 4-chlorothiophenol (2.88 g, 20 mmol), and K₂CO₃ (4.14 g, 30 mmol) were combined in DMF (50 mL) and stirred at 50° C. for 16 hours. The mixture was poured into water (100 mL) and extracted with ethyl acetate. The acetate layer was washed with water, brine, dried over anhydrous MgSO₄, filtered, and the filtrate concentrated under reduced pressure. The residue was purified by column chromatography to provide the title compound. ¹H NMR (300 MHz, DMSO-d₆) δ 7.21 (dd, J=4.5 Hz, 9 Hz, 1H), 7.50 (m, 5H), 7.79 (dd, J=3 Hz, 9 Hz, 1H), 10.25 (s, 1H); MS (DCI/NH₃) m/z 267 (M+H)⁺, 284 (M+NH₄)⁺.

### EXAMPLE 8B

### ethyl (2E)-3-{2-[(4-chlorophenyl)thio]-5-fluorophenyl}acrylate

The product from Example 8A and triethyl phosphonoacetate were processed as described in Example 1A to provide the title compound.

### EXAMPLE 8C

### methyl 3-{2-[(4-chlorophenyl)thio]-5-fluorophenyl}propanoate

The product from Example 8B was processed as described in Example 1B to provide the title compound.

### EXAMPLE 8D

### 3-{2-[(4-chlorophenyl)thio]-5-fluorophenyl}propanoic acid

The product from Example 8C was processed as described in Example 1C to provide the title compound.

### EXAMPLE 8E

### 3-{2-[(4-chlorophenyl)thio]-5-fluorophenyl}-N-(4-hydroxybutyl)propanamide

The product from Example 8D and 4-amino-1-butanol were processed as described in Example 1D to provide the title compound. ¹H NMR (300 MHz, DMSO-d₆) δ 1.38 (m, 4H), 2.34 (t, J=9 Hz, 2H), 2.93 (t, J=9 Hz, 2H), 3.01 (d, J=9 Hz, 2H), 3.36 (d, J=9 Hz, 2H), 4.36 (t, J=4.5 Hz, 1H), 7.13 (m, 3H), 7.26 (m, 1H), 7.52 (m, 3H), 8.78 (t, J=7 Hz, 1H); MS (DCI/NH₃) m/z 382 (M+H)⁺. Analysis calcd for C₁₉H₂₀ClFNO₂S: C, 59.76; H, 5.54; N, 3.67. Found: C, 59.73; H, 5.45; N, 3.73.

### EXAMPLE 9

### 3-{2-[(4-chlorophenyl)thio]phenyl}-N-(4-hydroxypentyl)propanamide

### EXAMPLE 9A

### 3-{2-[(4-chlorophenyl)thio]phenyl}acrylic acid

The product from Example 1A (4.8 g, 15 mmol) in 1,4-dioxane (30 mL) and ethanol (15 mL) was treated with 1N NaOH (20 mL) at 50° C. for 1 hour. The mixture was allowed to cool to room temperature, water was added, and the organics were removed under reduced pressure. The aqueous solution was acidified to pH 3 and filtered to provide the title compound as a solid. ¹H NMR (300 MHz, DMSO-d₆) 6.58 (d, J=15 Hz, 1H), 7.20 (d, J=9 Hz, 2H), 7.25 (d, J=9 Hz, 2H), 7.30 (m, 3H), 7.95 (m, 1H), 8.02 (d, J=15 Hz, 1H).

### EXAMPLE 9B

### 1-(3-{2-[(4-chlorophenyl)thio]phenyl}-2-propenoyl)-2-pyrrolidinone

The product from Example 9A (500 mg, 1.72 mmol) in methylene chloride (20 mL) at 0° C. was treated with a 2.0M methylene chloride solution of oxalyl chloride (0.86 mL, 1.72 mmol) and a catalytic drop of DMF. The mixture was allowed to gradually warm to room temperature over 1 hour. The mixture was recooled to 0° C. and treated sequentially with 2-pyrrolidinone (0.14 mL, 1.9 mmol) and triethylamine (0.48 mL, 3.4 mmol) via syringe. The mixure was allowed to warm to room temperature and stir for 16 hours. The mixture was treated with with 2N hydrochloric acid, diluted with ethyl acetate (300 mL), and the layers were separated. The ethyl acetate phases were combined, washed with 2N HCl, saturated aqueous NaHCO₃ (X2), brine, dried over MgSO₄, filtered, and the filtrate was concentrated under reduced pressure. The residue was purified by chromatography (silica gel, ethyl acetate:hexanes 1:2) to provide the title compound, mp 144-147° C. ¹H NMR (300 MHz, CD₂Cl₂) δ 1.95 (m, 2H), 2.52 (t, J=7.5 Hz, 2H), 3.76 (t, J=7.5 Hz, 2H), 7.09 (m, 2H), 7.18 (m, 2H), 7.30 (m, 4H), 7.70 (m, 1H), 7.78 (d, J=15 Hz, 1H, major rotamer), 8.19 (d, J=15 Hz, 1H, major rotamer), 8.30 (d, J=15 Hz, 1H minor rotamer); MS (APCI+) m/z 358 (M+H)⁺.

### EXAMPLE 9C

### 3-{2-[(4-chlorophenyl)thio]phenyl}-N-(4-hydroxypentyl)acrylamide

The product from Example 9B (393 mg, 1.10 mmol) in tetrahydrofuran (16 mL) at −78 ° C. solution was treated dropwise with methylmagnesium bromide (0.40 mL, 1.21 mmol). The mixture was allowed to slowly warm to ambient temperature and stirr for 16 hours. The mixture was quenched with 1N HCl solution and diluted with ethyl acetate. The layers were separated and the aqueous solution extracted with ethyl acetate (X2). The ethyl acetate phases were combined, dried over MgSO₄, filtered, and the filtrate concentrated under reduced pressure. The residue was purified by chromatography (silica gel, ethyl acetate:hexanes 1:1) to provide the title compound. ¹H NMR (300 MHz, CDCl₃) δ 8.05 (d, J=15 Hz, 1H), 7.59 (m, 1H), 7.22 (m, 7H), 6.30 (d, J=15 Hz, 1H), 5.84 (bm, 1H), 3.85 (m, 1H), 3.39 (m, 2H), 1.60 (m, 5H), 1.22 (d, J=6.0 Hz, 3H); MS (APCI+) m/z 376 (M+H)⁺.

### EXAMPLE 9D

### 3-{2-[(4-chlorophenyl)thio]phenyl}-N-(4-hydroxypentyl)propanamide

The product from Example 9C (31 mg, 0.083 mmol) in methanol (2 mL) at room temperature was treated with Mg shavings (22 mg, 0.91 mmol) and stirred for 16 hours. The mixture was concentrated under reduced pressure and the residue redissolved in a bi-phasic solution of 1N aqueous HCl and ethyl acetate. The layers were separated and the aqueous phase was extracted twice with ethyl acetate. The ethyl acetate phases were combined, dried over MgSO₄, filtered, and the filtrate concentrated under reduced pressure. The residue was purified by chromatography (silica gel, ethyl acetate:hexanes 3:1) to provide the title compound. ¹H NMR (300 MHz, CDCl₃) δ 7.20 (m, 8H), 5.50 (bm, 1H), 3.79 (m, 1H), 3.23 (m, 2H), 3.10 (t, J=8.1 Hz, 2H), 2.43 (t, J=8.1 Hz, 2H), 1.58 (m, 3H), 1.40 (m, 2H), 1.18 (d, J=6.0 Hz, 3H); MS (APCI+) m/z 378 (M+H)⁺. FAB HRMS m/z for C₂₀H₂₅NO₂ClS (M+H)⁺: calcd 378.1295; found 378.1301.

### EXAMPLE 10

### 3-{2-[(4-chlorophenyl)thio]phenyl}-N-methyl-N-(1-methyl-4-piperidinyl)butanamide

### EXAMPLE 10A

### 1-{2-[(4-chlorophenyl)thio]phenyl}ethanone

2-Nitroacetophenone (2.5 g, 15.13 mmol), anhydrous K₂CO₃ (4.18 g, 30.27 mmol) and 4-chlorothiophenol (2.4 g, 16.65 mmol) in DMF (35 mL) were combined and stirred at 50° C. for 12 hours. The mixture was poured into water and extracted with ethyl acetate. The acetate extract was washed with water, brine, dried with anhydrous MgSO₄, filtered, and the filtrate concentrated under reduced pressure. The residue was purified by column chromatography (silica gel, 9:1 hexane:ethyl acetate) to provide the title compound. MS ((DCI/NH₃) m/z 263 (M+NH₄)⁺.

### EXAMPLE 10B

### ethyl 3-{2-[(4-chlorophenyl)thio]phenyl}-2-butenoate

Triethyl phosphonoacetate (0.8 mL, 4 mmol) in THF (25 mL) was treated with 60% NaH (160 mg, 4 mmol) and then treated with the product from Example 10A (1.7 g, 6 mmol). After stirring at room temperature for 16 hours, the mixture was concentrated under reduced pressure, 1N HCl was added to attain pH 2-3, and the mixture was extracted with ethyl acetate. The acetate layer was washed with water, brine, dried with MgSO₄ filtered, and the filtrate concentrated under reduced pressure to afford 1.46 g of residue as mixture of Z and E isomers. The crude mixture was purified by column chromatography (silica gel, hexane:ethyl acetate, 9:1) to provide the E isomer, the Z isomer, and a mixture of Z and E isomers. ¹H NMR (300 MHz, DMSO-d₆) for E isomer; δ 1.21 (t, J=7 Hz, 3H), 2.39 (s, 3H), 4.11 (q, J=7 Hz, 2H), 5.62. (s, 1H), 7.23 (dd, J=9 Hz and 3 Hz, 2H), 7.29 (m, 2H), 7.39 (m, 4H); MS (APCI+) m/z 333 (M+H)⁺. For Z isomer; δ 0.98 (t, J=7 Hz, 3H), 2.09 (s, 3H), 3.84 (q, J=7 Hz, 2H), 6.0 (s, 1H), 7.16 (dd, J=9 Hz and 3 Hz, 2H), 7.22 (m, 2H), 7.35 (m, 4H); MS (APCI+) m/z 333 (M+H)⁺.

### EXAMPLE 10C

### 3-{2-[(4-chlorophenyl)thio]phenyl}-2-butenoic acid

The Z isomer, E isomer and mixture of Z and E isomers from Example 10B were combined (0.3 g, 0.94 mmol) in methanol:1,4-dioxane (2:1, 25 mL) and treated with 1N NaOH (8 mL) at 80° C. for 2 hours. The mixture was allowed to cool to room temperature, water was added and the organics were removed under reduced pressure. The aqueous layer was acidified with 10% citric acid and extracted with ethyl acetate. The ethyl acetate layers were combined, dried over MgSO₄, filtered, and the filtrate concentrated to provide the title compound as a solid. ¹H NMR (300 MHz, DMSO-d₆) δ 2.12 (s, 1.5H), 2.41 (s, 1.5 H), 5.61. (s, 0.5H), 5.93 (s, 0.5H), 7.25 (m, 8H); MS (DCI) m/z 307 (M+H)⁺.

### EXAMPLE 10D

### 3-{2-[(4-chlorophenyl)thio]phenyl}-N-methyl-N-(1-methyl-4-piperidinyl)-2-butenamide

The product from Example 10C (88 mg, 0.28 mmol), EDCI (109 mg, 0.57 mmol), 1-hydroxybenzotriazole (36 mg, 0.25 mmol), and 1-methyl-4-(methylamino)piperidine (0.08 mL, 0.5 mmol) were combined in dichloromethane:THF (4 mL:2 mL) and stirred at room temperature for 18 hours. The mixture was concentrated under reduced pressure and the residue was partitioned between water and ethyl acetate. The ethyl acetate extract was washed with water, brine, dried with MgSO₄, filtered, and the filtrate concentrated under reduced pressure. The residue was purified by column chromatography (silica gel, CH₂Cl₂:methanol, 9.5:0.5) to provide the title compound. ¹H NMR (300 MHz, CD3OD) δ 1.3 (m, 2H), 1.5 (m, 1H), 1.7 (m, 3H), 2.12 (m, 5H), 2.26 (m, 3H), 2.62 (s, 1H), 2.88 (m, 4H), 3.86 (m, 0.5H), 4.08 (m, 0.5H), 6.28 (m, 1H), 7.25(m, 8H); MS (DCI) m/z 415 (M+H)⁺.

### EXAMPLE 10E

### 3-{2-[(4-chlorophenyl)thio]phenyl}-N-methyl-N-(1-methyl-4-piperidinyl)butanamide

The product from Example 10D (96 mg, 0.23 mmol) in methanol was treated with Mg turnings (55 mg, 2.3 mmol) and stirred at room temperature for 55 hours. The mixture was treated with 1N HCl, stirred for 15 minutes, and neutralized with NaHCO₃. The mixture was concentrated under reduced pressure and the agueous solution extracted with ethyl acetate. The ethyl acetate extract was washed with brine, dried with MgSO₄, filtered, and the filtrate was concentrated under reduced pressure. The residue was purified by column chromatography (silica gel, 9.5:0.5 CH₂Cl₂:methanol) to provide the title compound. ¹H NMR (300 MHz, CD₃OD) δ 1.25 (m, 2H), 1.5 (m, 1H), 1.7 (m, 3H), 2.12 (m, 5H), 2.26 (m, 2H), 2.58 (m, 1H), 2.81 (m, 5H), 3.96 (m, 0.5H), 4.3 (m, 0.5H), 7.1 (m, 1H), 7.3(m, 6H); MS (DCI) m/z 417 (M+H)⁺.

### EXAMPLE 11

### 3-{2-[(4-chlorophenyl)thio]phenyl}-N-[2-(4-pyridinyl)ethyl]butanamide

### EXAMPLE 11A

### 3-{2-[(4-chlorophenyl)thio]phenyl}-N-[2-(4-pyridinyl)ethyl]-2-butenamide

The product from Example 10C and 4-(2-aminoethyl)pyridine were processed as described in Example 10D to provide the title compound.

### EXAMPLE 11B

### 3-{2-[(4-chlorophenyl)thio]phenyl}-N-[2-(4-pyridinyl)ethyl]butanamide

The product from Example 11A was processed as described in Example 10E to provide the title compound. ¹H NMR (300 MHz, CD₃OD) δ 1.13 (d, J=6 Hz, 3H), 2.4 (m, 3H), 2.75 (m, 3H), 3.4 (m, 3H), 3.94 (m, 1H), 7.25 (m, 11H), 8.38 (m, 2H); MS (DCI) m/z 411 (M+H)⁺.

### EXAMPLE 12

### 3-{2-[(4-chlorophenyl)thio]phenyl}-N-[4-(methoxyamino)butyl]propanamide

### EXAMPLE 12A

### 4-[(3-{2-[(4-chlorophenyl)thio]phenyl}propanoyl)amino]butyl methanesulfonate

The product from Example 1D (330 mg, 0.91 mmol) in pyridine (15 mL) was treated with methanesulfonyl chloride (0.5 mL, 5 mmol) at 0° C. After 3 hours, the mixture was poured into ice/10% citric acid and extracted with ethyl acetate. The ethyl acetate layer was washed with water, brine, dried with MgSO₄, filtered, and the filtrate concentrated under reduced pressure to provide the title compound which was used in the next reaction without further purification.

### EXAMPLE 12B

### 3-{2-[(4-chlorophenyl)thio]phenyl}-N-[4-(methoxyamino)butyl]propanamide

The product from Example 12A (441 mg, 1 mmol), O-methylhydroxylamine hydrochloride (250 mg, 3 mmol), and Cs₂CO₃ (650 mg, 2 mmol) were combined in ethanol (30 mL) and refluxed for 12 to 24 hours. The mixture was allowed to cool to room temperature and concentrated under reduced pressure. The residue was purified by column chromatography (silica gel, ethyl acetate) to provide the title compound. ¹H NMR (300 MHz, DMSO-d₆) δ 1.35 (m, 4H), 2.34 (m, 2H), 2.70 (m, 2H), 2.91 (m, 2H), 3.00 (m, 2H), 3.37 (s, 3H), 6.46 (t, J=6 Hz, 1H), 7.14 (m, 2H), 7.32 (m, 6H), 7.80 (t, J=6 Hz, 1H); MS (DCI/NH₃) m/z 393 (M+H)⁺.

### EXAMPLE 13

### 3-{2-[(4-chlorophenyl)thio]phenyl}-N-[4-(methylamino)butyl]propanamide

The product from Example 12A and methanamine hydrochloride were processed as described in Example 12B to provide the title compound. ¹H NMR (300 MHz, DMSO_{d}₆) δ 1.35 (m, 4H), 2.25 (s, 3H), 2.33 (m, 2H), 2.37 (t, J=7 Hz, 2H), 2.93 (t, J=7 Hz, 2H), 3.01 (m, 2H), 7.16 (d, J=9 Hz, 2H), 7.32 (m, 6H), 7.81 (t, J=6 Hz, 1H); MS (DCI/NH₃) m/z 377 (M+H)⁺.

### EXAMPLE 14

### 3-{2-[(4-chlorophenyl)thio]phenyl}-N-[5-(methylamino)pentyl]propanamide

### EXAMPLE 14A

### 5-[(3-{2-[(4-chlorophenyl)thio]phenyl}propanoyl)amino]pentyl methanesulfonate

The product from Example 3 and methanesulfonyl chloride were processed as described in Example 12A to provide the title compound.

### EXAMPLE 14B

### 3-{2-[(4-chlorophenyl)thio]phenyl}-N-[5-(methylamino)pentyl]propanamide

The product from Example 14A and methanamine hydrochloride were processed as described in Example 12B to provide the title compound. ¹H NMR (300 MHz, DMSO-d₆) δ 1.23 (m, 2H), 1.38 (m, 4H), 2.27 (s, 3H), 2.34 (t, J=7 Hz, 2H), 2.41 (t, J=7 Hz, 2H), 2.93 (t, J=7 Hz, 2H), 3.01 (m, 2H), 7.17 (d, J=9 Hz, 2H), 7.32 (m, 6H), 7.78 (t, J=6 Hz, 1H); MS (DCI/NH₃) m/z 391 (M+H)⁺. FAB HRMS m/z for C₂₀H₂₄NO₂S (M+H)⁺: calcd 342.1528, found 342.1537.

### EXAMPLE 15

### (2E)-N-(4-hydroxycyclohexyl)-3-{2-[(4-methylphenyl)thio]-3-pyridinyl}acrylamide

### EXAMPLE 15A

### diethyl 2-[(4-hydroxycyclohexyl)amino]-2-oxoethylphosphonate

(Diethoxyphosphoryl)acetic acid (1.5 g, 7.5 mmol), dicyclohexylcarbodimide, N′-methyl polystyrene (12.0 g, 23.0 mmol), and HOBt (1.04 g, 7.7 mmol) were combined in DMA/DCM (1:1, 120 mL) and shaken at ambient temperature for 15 minutes. The mixture was treated with 4-aminocyclohexanol (1.21 g, 10.5 mmol) in DMA/DCM (1:1, 50 mL) and shaken at room temperature for 18 hours. The mixture was then treated with trisamine polystyrene (12.0 g, 48 mmol) and shaken at room temperature for an additional 4 hours. The mixture was filtered and the resin was washed with DCM (2×10 mL). The combined filtrates were concentrated under reduced pressure to provide the title compound which was used in the next reaction without further purification. ¹H NMR (300 MHz, CDCl₃) δ 1.25 (m, 2H), 1.37 (t, J=7 Hz, 6H), 1.40 (m, 2H), 2.00 (m, 4H), 2.80 (s, 1H), 2.90 (s, 1H), 3.60 (m, 1H), 3.78 (m, 1H), 4.13 (q, J=7 Hz, 4H), 6.60 (m, 1H).

### EXAMPLE 15B

### (2E)-N-(4-hydroxycyclohexyl)-3-{2-[(4-methylphenyl)thio]-3-pyridinyl}acrylamide

The product from Example 15A (44 mg, 0.15 mmol) in anhydrous THF (1.5 mL) was treated with LDA (2.0 M, 0.35 mL, 0.7 mmol) at room temperature. After 30 minutes, the mixture was treated with 2-[(4-methylphenyl)thio]nicotinaldehyde (23 mg, 0.1 mmol) in anhydrous THF (0.5 mL) and shaken at room temperature for 1 hour. The mixture was then treated with water (0.2 mL) and concentrated under reduced pressure. The residue was purified by preparative HPLC to provide the title compound. ¹H NMR (300 MHz, DMSO-d₆) δ 1.20 (m, 4H), 1.80 (m, 4H), 2.38 (s, 3H), 3.40 (m, 1H), 3.60 (m, 1H), 6.62 (d, J=15 Hz, 1H), 7.22 (m, 3H), 7.39 (d, J=6 Hz, 2H), 7.67 (d, J=15 Hz, 1H), 7.87 (d, J=3 Hz, 1H), 8.10 (d, J=3 Hz, 1H), 8.28 (m, 1); MS (ESI APCI+) m/z 369 (M+H)⁺.

### EXAMPLE 16

### (2E)-3-{2-[(4-chlorophenyl)thio]-3-pyridinyl}-N-(4-hydroxycyclohexyl)acrylamide

The product from Example 15A and 2-[(4-chlorophenyl)thio]nicotinaldehyde were processed as described in Example 15B to provide the title compound. ¹H NMR (300 MHz, DMSO-d₆) δ 1.20 (m, 4H), 1.80 (m, 4H), 3.40 (m, 1H), 3.60 (m, 1H), 4.56 (br s, 1H), 6.62 (d, J=15 Hz, 1H), 7.25 (m, 1H), 7.49 (m, 4H), 7.63 (d, J=15 Hz, 1H), 7.92 (d, J=3 Hz, 1H), 8.10 (d, J=3 Hz, 1H), 8.35 (m, 1); MS (ESI APCI+) m/z 389 (M+H)⁺.

### EXAMPLE 17

### (2E)-3-{2-[(2,4-dichlorophenyl)thio]-3-pyridinyl}-N-(4-hydroxycyclohexyl)acrylamide

The product from Example 15A and 2-[(2,4-dichlorophenyl)thio]nicotinaldehyde were processed as described in Example 15B to provide the title compound. ¹H NMR (300 MHz, DMSO-d₆) δ 1.20 (m, 4H), 1.80 (m, 4H), 3.40 (m, 1H), 3.60 (m, 1H), 6.64 (d, J=15 Hz, 1H), 7.31 (m, 1H), 7.48 (d, J=6 Hz, 1H), 7.63 (m, 2H), 7.80 (s, 1H), 7.96 (d, J=4 Hz, 1H), 8.13 (d, J=4 Hz, 1H), 8.36 (m, 2H); MS (ESI APCI+) m/z 424 (M+H)⁺.

### EXAMPLE 18

### (2E)-3-{2-[(4-bromophenyl)thio]-3-pyridinyl}-N-(4-hydroxycyclohexyl)acrylamide

The product from Example 15A and 2-[(4-bromophenyl)thio]nicotinaldehyde were processed as described in Example 15B to provide the title compound. ¹H NMR (300 MHz, DMSO-d₆) δ 1.20 (m, 4H), 1.80 (m, 4H), 3.40 (m, 1H), 3.60 (m, 1H), 4.57 (br s, 1H), 6.62 (d, J=15 Hz, 1H), 7.33 (m, 1H), 7.41 (m, 2H), 7.62 (m, 3H), 7.93 (d, J=4 Hz, 1H), 8.13 (d, J=4 Hz, 1H), 8.37 (m, 1H); MS (ESI APCI+) m/z 434 (M+H)⁺.

### EXAMPLE 19

### (2E)-N-(4-hydroxycyclohexyl)-3-[2-(phenylthio)-3-pyridinyl]acrylamide

The product from Example 15A and 2-(phenylthio)nicotinaldehyde were processed as described in Example 15B to provide the title compound. ¹H NMR (300 MHz, DMSO-d₆) δ 1.20 (m, 4H), 1.80 (m, 4H), 3.40 (m, 1H), 3.60 (m, 1H), 4.55 (br s, 1H), 6.61 (d, J=15 Hz, 1H), 7.28 (m, 1H), 7.43 (m, 5H), 7.72 (d, J=15 Hz, 1H), 7.92 (d, J=5 Hz, 1H), 8.10 (d, J=5 Hz, 1H), 8.33 (m, 1H); MS (ESI APCI+) m/z 355 (M+H)⁺.

### EXAMPLE 20

### (2E)-3-{2-[(2-chlorophenyl)thio]-3-pyridinyl}-N-(4-hydroxycyclohexyl)acrylamide

The product from Example 15A and 2-[(2-chlorophenyl)thio]nicotinaldehyde were processed as described in Example 15B to provide the title compound. ¹H NMR (300 MHz, DMSO-d₆) δ 1.20 (m, 4H), 1.80 (m, 4H), 3.40 (m, 1H), 3.60 (m, 1H), 4.60 (br s, 1H), 6.65 (d, J=15 Hz, 1H), 7.28 (m, 1H), 7.38-7.44 (m, 2H), 7.58 (d, J=7 Hz, 1H), 7.62 (d, J=7 Hz, 2H), 7.68 (d, J=15 Hz, 1H), 7.96 (d, J=4 Hz, 1H), 8.12 (d, J=4 Hz, 1H), 8.36 (m, 1H) MS (ESI APCI+) m/z 389 (M+H)⁺.

### EXAMPLE 21

### (2E)-N-(2-hydroxycyclohexyl)-3-[2-(isobutylthio)phenyllacrylamide

### EXAMPLE 21A

### diethyl 2-[(2-hydroxycyclohexyl)amino]-2-oxoethylphosphonate

(Diethoxyphosphoryl)acetic acid and 2-aminocyclohexanol were processed as described in Example 15A to provide the title compound.

### EXAMPLE 21B

### (2E)-N-(2-hydroxycyclohexyl)-3-[2-(isobutylthio)phenyl]acrylamide

The product from Example 21A and 2-(isobutylthio)benzaldehyde were processed as described in Example 15B to provide the title compound. ¹H NMR (300 MHz, DMS)-d₆) δ 0.99 (d, J=7 Hz, 6H), 1.20 (m, 4H), 1.60 (m, 2H), 1.80 (m, 1H), 1.85 (m, 2H), 2.82 (d, J=6 Hz, 2H), 3.25 (m, 1H), 3.55 (m, 1H), 4.60 (br s, 1H), 6.60 (d, J=15 Hz, 1H), 7.20 (m, 1), 7.38 (m, 1H), 7.42 (d, J=5 Hz, 1H), 7.57 (d, J=5 Hz, 1H), 7.83 (d, J=15 Hz, 1H), 7.99 (d, J=4 Hz, 1H); MS (ESI APCI+) m/z 334 (M+H)⁺.

### EXAMPLE 22

### (2E)-3-[2-(cyclopentylthio)phenyl]-N-(2-hydroxycyclohexyl)acrylamide

The product from Example 21A and 2-(cyclopentylthio)benzaldehyde were processed as described in Example 15B to provide the title compound. ¹H NMR (300 MHz, DMSO-d₆) δ 1.20 (m, 4H), 1.42-1.60 (m, 5H), 1.72 (m, 2H), 1.82 (m, 2H), 2.02 (m, 3H), 3.25 (m, 1H), 3.55 (m, 1H), 3.65 (m, 1H), 4.55 (br s, 1H), 6.60 (d, J=15 Hz, 1H), 7.20-7.60 (m, 4H), 7.81 (d, J=15 Hz, 1H), 7.98 (d, J=4 Hz, 1H); MS (ESI APCI+) m/z 346 (M+H)⁺.

### EXAMPLE 23

### (2E)-3-[2-(cyclohexylthio)phenyl]-N-(2-hydroxycyclohexyl)acrylamide

The product from Example 21A and 2-(cyclohexylthio)benzaldehyde were processed as described in Example 15B to provide the title compound. ¹H NMR (300 MHz, DMSO-d₆) δ 1.15-1.40 (m, 8H), 1.50-1.80 (m, 6H), 1.88 (m, 4H), 3.20 (m, 1H), 3.35 (m, 1H), 3.55 (m, 1H), 4.59 (br s, 1H), 6.60 (d, J=15 Hz, 1H), 7.22-7.55 (m, 3H), 7.59 (d, J=5 Hz, 1H), 7.93 (d, J=15 Hz, 1H), 7.97 (d, J=4 Hz, 1H); MS (ESI APCI+) m/z 360 (M+H)⁺.

### EXAMPLE 24

### (2E)-N-(4-hydroxycyclohexyl)-3-[2-(isopropylthio)phenyl]acrylamide

The product from Example 15A (15 mg, 0.05 mmol) in anhydrous THF (1.0 mL) was treated with LDA (2.0 M, 175 ul, 0.25 mmol) at room temperature. After 10 minutes, the mixture was treated with 2-(isopropylthio)benzaldehyde (8 mg, 0.04 mmol) in anhydrous THF (0.5 mL) and shaken at room temperature for 1 hour. The mixture was then treated with water (0.3 mL) and concentrated under reduced pressure. The residue was purified by preparative HPLC to provide the title compound. ¹H NMR (500 MHz, DMSO-d₆) 1.22 (m, 10H), 1.82 (m, 4H), 2.85 (s, 6H), 3.40 (m, 2H), 3.58 (m, 1H), 4.55 (m, 1H), 6.55 (d, J=15 Hz, 1H), 7.38 (m, 2H), 7.52 (d, J=4 Hz, 1H), 7.58 (d, J=4 Hz, 1H), 7.90 (d, J=15 Hz, 1H), 8.00 (m, 1H), MS (ESI APCI−) m/z 318 (M−H)⁻.

### EXAMPLE 25

### (2E)-3-[2-(cyclopentylthio)phenyl]-N-(4-hydroxycyclohexyl)acrylamide

The product from Example 15A and 2-(cyclopentylthio)benzaldehyde were processed as described in Example 15B to provide the title compound. ¹H NMR (500 MHz, DMSO-d₆) 1.22 (m, 4H), 1.52 (m, 2H), 1.58 (m, 2H), 1.75 (m, 2H), 1.82 (m, 4H), 2.05 (m, 2H), 3.38 (m, 1H), 3.58 (m, 1H), 3.62 (m, 1H), 4.55 (d, J=3 Hz, 1H), 6.55 (d, J=15 Hz, 1H), 7.28 (t, J=3 Hz, 1H), 7.35 (t, J=3 Hz, 1H), 7.52 (d, J=4 Hz, 1H), 7.58 (d, J=4 Hz, 1H), 7.92 (d, J=15 Hz, 1H), 7.98 (d, J=3 Hz, 1H), MS (ESI APCI+) m/z 346 (M+H)⁺.

### EXAMPLE 26

### (2E)-3-[2-(cyclohexylthio)phenyl]-N-(4-hydroxycyclohexyl)acrylamide

The product from Example 15A and 2-(cyclohexylthio)benzaldehyde were processed as described in Example 15B to provide the title compound. ¹H NMR (500 MHz, DMSO-d₆) 1.22 (m, 6H), 1.30 (m, 4H), 1.55 (m, 1H), 1.70 (m, 2H), 1.83 (m, 5H), 3.20 (m, 1H), 3.38 (m, 1H), 3.60 (m, 1H), 4.55 (m, 1H), 6.54 (d, J=15 Hz, 1H), 7.32 (m, 2H), 7.52 (d, J=4 Hz, 1H), 7.58 (d, J=4 Hz, 1H), 7.92 (d, J=15 Hz, 1H), 7.98 (br, 1H); MS (ESI APCI+) m/z 360 (M+H)⁺.

### EXAMPLE 27

### (2E)-N-(4-hydroxycyclohexyl)-3-[2-(pentylthio)phenyl]acrylamide

The product from Example 15A and 2-(pentylthio)benzaldehyde were processed as described in Example 15B to provide the title compound as a trifluoroacetic acid salt. ¹H NMR (500 MHz, DMSO-d₆) 0.83 (t, J=5, 3H), 1.25 (m, 6H), 1.38 (m, 2H), 1.58 (m, 2H), 1.82 (m, 2H), 2.96 (m, 2H), 3.20 (m, 3H), 3.60 (m, 1H), 6.58 (d, J=15 Hz, 1H), 7.25 (t, J=3 Hz, 1H), 7.35 (t, J=3 Hz, 1H), 7.43 (d, J=4 Hz, 1H), 7.53 (d, J=4 Hz, 1H), 7.82 (d, J=15 Hz, 1H), 7.98 (d, J=3 Hz, 1H); MS (ESI APCI+) m/z 348 (M+H)⁺.

### EXAMPLE 28

### (2E)-3-{2-[(4-chlorophenyl)thio]-6-methyl-3-pyridinyl}-N-(4-hydroxycyclohexyl)acrylamide

### EXAMPLE 28A

### 2-[(4-chlorophenyl)thio]-6-methylnicotinonitrile

2-Chloro-3-cyano-6-methylpyridine (1 g, 6.5 mmol), K₂CO₃ (1.4 g, 10 mmol), and 4-chlorothiophenol (1 g, 7 mmol) were combined in DMF (30 mL) and heated at 50° C. for 16 hours. The mixture was allowed to cool to room temperature, poured into water (100 mL), and extracted with ethyl acetate. The acetate layer was washed with water, brine, dried with MgSO₄, filtered, and the filtrate concentrated under reduced pressure. The residue was treated with hexane and the precipitated solid was filtered to provide the title compound. ¹H NMR (300 MHz, DMSO-d6) δ 2.38 (s, 3H), 7.26 (m, 1H), 7.55 (m, 4H), 8.18 (d, J=9 Hz, 1H); MS (DCI/NH₃) m/z 261 (M+H)⁺.

### EXAMPLE 28B

### 2-[(4-chlorophenyl)thio]-6-methylnicotinaldehyde

The product from Example 28A (1.3 g, 5 mmol) in THF (30 mL) was treated with DIBAL (1N solution in THF) (7.5 mL, 7.5 mmol) and left at room temperature for 14 hours. The mixture was treated with additional DIBAL (10 mL, 10 mmol), left at room temperature for another 14 hours, and then heated at 50° C. for 1 hour. The mixture was allowed to cool to room temperature and treated with 10% citric acid to pH 5. The mixture was concentrated under reduced pressure and the remaining aqueous solution was extracted with ethyl acetate. The organic layers were combined, dried with MgSO₄, filtered, and the filtrate concentrated under reduced pressure. The residue was purified by column chromatography (silica gel, 2:1 hexane:ethyl acetate to provide the title compound. MS (DCI/NH₃) m/z 264 (M+H)⁺.

### EXAMPLE 28C

### (2E)-3-{2-[(4-chlorophenyl)thio]-6-methyl-3-pyridinyl}-N-(4-hydroxycyclohexyl)acrylamide

The product from Example 28B and the product from Example 15A were processed as described in Example 15B to provide the title compound. ¹H NMR (300 MHz, DMSO-d6) δ 1.22 (m, 4H), 1.81 (m, 4H), 2.3 (s, 3H), 3.38 (m, 1H), 3.59(S, 1H), 4.52 (d, J=4.5 Hz, 1H), 6.59 (D, J=15 Hz, 1H), 7.17 (d, J=9 Hz, 1H), 7.45 (s, 4H), 7.64 (d, J=15 Hz, 1H), 7.81 (d, J=9 Hz, 1H), 8.04 (d, J=7.5 Hz, 1H); MS (DCI) m/z 403 (M+H)⁺.

### EXAMPLE 29

### (2E)-3-{2-[(2,4-dichlorophenyl)thio]phenyl}-N-(4-hydroxybutyl)acrylamide

### EXAMPLE 29A

### 2-[(2,4-dichlorophenyl)thio]benzaldehyde

2-Nitrobenzaldehyde (1.51 g, 10 mmol), anhydrous K₂CO₃ (2.76 g, 20 mmol), and 2,4-dichlorothiophenol (2.1 g, 11 mmol) were combined in DMF (35 mL) and stirred at 50° C. under N₂ for 12 hours. The mixture was then poured into water and extracted with EtOAc. The acetate extract was washed with water, brine, dried with anhydrous MgSO₄, filtered, and the filtrate was concentrated under reduced pressure. The residue was purified by column chromatography (silica gel, 9:1 hexane:EtOAc) to provide the title compound. MS ((DCI-NH₃) m/z 300 (M+NH₄)⁺.

### EXAMPLE 29B

### ethyl (2E)-3-{2-[(2,4-dichlorophenyl)thio]phenyl}acrylate

Triethyl phosphonoacetate (1.24 mL, 6 mmol) in THF (25 mL) was treated with 60% NaH (240 mg, 6 mmol) and then treated with the product from Example 29A (1.7 g, 6 mmol). The mixture was stirred at room temperature for 16 hours and then concentrated under reduced pressure. The mixture was treated with 10% citric acid and extracted with ethyl acetate. The acetate layer was washed with water, brine, dried with MgSO₄, filtered, and the filtrate concentrated under reduced pressure to provide the title compound. ¹H NMR (300 MHz, CDCl₃) δ 1.30 (7, J=7 Hz, 3H), 4.24 (q, J=7 Hz, 2H), 6.38 (d, J=15 Hz, 1H), 6.73 (d, J=9 Hz, 1H), 7.05 (dd, J=9 Hz and 3 Hz, 1H), 7.42 (m, 4H), 7.70 (m, 1H), 8.15 (d, J=15 Hz, 1H); MS (APCI+) m/z 353 (M+H)⁺.

### EXAMPLE 29C

### (2E)-3-{2-[(2,4-dichlorophenyl)thio]phenyl}acrylic acid

The product from Example 29B (1.76 g, 5 mmol) in 1,4-dioxane (15 mL) and methanol (25 mL) was treated with 1N NaOH (6 mL) at room temperature and stirred for 24 hours. The mixture was treated with water and the organics were removed under reduced pressure. The water solution was acidified to pH 3 and extracted with ethyl acetate. The acetate extract layer was separated, dried with MgSO₄, filtered, and the filtrate concentrated under reduced pressure to provide the title compound. ¹H NMR (300 MHz, CDCl₃) δ 6.56 (d, J=15 Hz, 1H), 6.72 (d, J=9 Hz, 1H), 7.34 (dd, J=9 Hz, 3 Hz, 1H), 7.53 (m, 3H), 7.74 (d, J=3 Hz, 1H), 7.92 (d, J=15 Hz, 1H), 8.00 (m, 1H), 12.53 (br s, 1H).

### EXAMPLE 29D

### (2E)-3-{2-[(2,4-dichlorophenyl)thio]phenyl}-N-(4-hydroxybutyl)acrylamide

The product from Example 29C (648 mg, 2 mmol), N-hydroxysuccinimide (230 mg, 2 mmol), and EDCI (490 mg, 2.5 mmol) were combined in CHCl₃ (30 mL) and stirred at ambient temperature for 30 minutes. The mixture was treated with 4-amino-1-butanol (223 mg, 2.5 mmol) and stirred at room temperature for 10 hours. The mixture was concentrated under reduced pressure and the residue was partitioned between water and ethyl acetate. The acetate layer was separated, washed with water, brine, dried with MgSO₄, filtered, and the filtrate concentrated under reduced pressure. The residue was purified by chromatography (silica gel, ethyl acetate) to provide the title compound. mp 68-71° C.; ¹H NMR (300 MHz, CDCl₃) δ 1.44 (m, 4H), 3.14 (q, J=7 Hz, 2H), 3.40 (q, J=7 Hz, 2H), 4.40 (t, J=7 Hz, 1H), 6.63 (d, J=15 Hz, 1H), 6.69 (d, J=9 Hz, 1H), 7.33 (dd, J=9 Hz, 3 Hz, 1H), 7.48 (m, 2H), 7.57 (m, 1H), 7.74 (m, 1H), 7.90 (m, 2H), 8.15 (t, J=7 Hz, 1H); MS (APCI+) m/z 396 (M+H)⁺. Analysis calcd for C₁₉H₁₉Cl₂NO₂S: C, 57.68; H, 4.83; N, 3.53. Found: C, 57.25, H, 4.90; N, 3.41.

### EXAMPLE 30

### (2E)-3-{2-[(4-chlorophenyl)thio]phenyl}-N-(4-hydroxycyclohexyl)acrylamide

The product from Example 9A and 4-aminocyclohexanol were processed as described in Example 1D to provide the title compound. mp 86-88° C. ¹H NMR (300 MHz, DMSO-d₆) δ 1.20 (m, 4H), 1.80 (m, 4H), 3.48 (m, 1H), 3.55 (m, 1H), 4.52 (d, J=5 Hz, 1H), 6.60 (d, J=15 Hz, 1H), 7.18 (d, 2H), 7.42 (m, 5H), 7.70 (d, J=9 Hz, 1H), 7.81 (d, J=15 Hz, 1H), 8.00 (d, J=7 Hz, 1H); MS (ESI APCI+) m/z 388 (M+H)⁺. Analysis calcd for C₂₁H₂₂ClNO₂S: C, 65.02; H, 5.72; N, 3.61. Found: C, 64.72; H, 5.86; N, 3.47.

### EXAMPLE 31

### (2E)-3-{2-[(4-chlorophenyl)thio]phenyl}-N-(5-hydroxy-1,5-dimethylhexyl)acrylamide

The product from Example 9A and 6-amino-2-methyl-2-heptanol were processed as described in Example 1D to provide the title compound. ¹H NMR (300 MHz, DMSO-d₆) δ 1.05 (d+s, 9H), 1.32 (m, 6H), 3.83 (m, 1H), 4.05 (s, 1H), 6.60 (d, J=15 Hz, 1H), 7.18 (d, J=9 Hz, 2H), 7.42 (m, 5H), 7.72 (d, J=9 Hz, 1H), 7.83 (d, J=15 Hz, 1H), 7.97 (d, J=7 Hz, 1H); MS (ESI APCI+) m/z 418 (M+H)⁺; (ESI APCI−) m/z 452 (M+Cl. Analysis calcd for C₂₃H₂₈ClNO₂S.0.25H₂O: C, 65.40; H, 6.77; N, 3.32. Found: C, 65.31; H, 6.79; N, 3.25.

### EXAMPLE 32

### (2E)-3-{2-[(4-chlorophenyl)thio]phenyl}-N-ethylacrylamide

The product from Example 9A and ethanamine were processed as described in Example 15A to provide the title compound. ¹H NMR (300 MHz, DMSO-d₆) δ 1.08 (t, J=7 Hz, 3H), 3.18 (q, J=7 Hz, 2H), 6.58 (d, J=15 Hz, 1H), 7.19 (d, J=9 Hz, 2H), 7.42 (m, 5H), 7.73 (d, J=4 Hz, 1H), 7.82 (d, J=15 Hz, 1H), 8.12 (m, 1H); MS (ESI APCI+) m/z 318 (M+H)⁺.

### EXAMPLE 33

### (2E)-N-butyl-3-{2-[(4-chlorophenyl)thio]phenyl}acrylamide

The product from Example 9A and 1-butanamine were processed as described in Example 15A to provide the title compound. ¹H NMR (300 MHz, DMSO-d₆) δ 0.83 (t, J=7 Hz, 3H), 1.22 (m, 2H), 1.40 (m, 2H), 3.15 (m, 2H), 6.60 (d, J=15 Hz, 1H), 7.19 (d, J=9 Hz, 2H), 7.42 (m, 5H), 7.73 (d, J=4 Hz, 1H), 7.82 (d, J=15 Hz, 1H), 8.12 (m, 1H); MS (ESI APCI+) m/z 346 (M+H)⁺.

### EXAMPLE 34

### (2E)-3-{2-[(4-chlorophenyl)thio]phenyl}-N-(4-hydroxybutyl)acrylamide

The product from Example 9A and 4-amino-1-butanol were processed as described in Example 1D to provide the title compound. ¹H NMR (300 MHz, CDCl₃) δ 1.44 (m, 4H), 3.41 (q, J=7 Hz, 2H), 3.70 (t, J=7 Hz, 2H), 5.85 (brs, 1H), 6.33 (d, J=15 Hz, 1H), 7.13 (d, J=9 Hz, 2H), 7.38 (m, 5H), 7.57 (m, 1H), 8.05 (d, J=15 Hz, 1H); MS (DCI-NH₃) m/z 362 (M+H)⁺.

### EXAMPLE 35

### (2E)-3-{2-[(4-chlorophenyl)thio]phenyl}-N-(5-hydroxypentyl)acrylamide

The product from Example 9A and 5-amino-1-pentanol were processed as described in Example 1D to provide the title compound. ¹H NMR (300 MHz, DMSO-d₆) δ 1.31 (m, 2H), 1.45 (m, 4H), 3.14 (q, J=7 Hz, 2H), 3.39 (q, J=7 Hz, 2H), 4.36 (t, J=7 Hz, 1H), 6.61 (d, J=15 Hz, 1H), 7.17 (d, J=9 Hz, 2H), 7.45 (m, 5H), 7.72 (d, J=9 Hz, 1H), 7.82 (d, J=15 Hz, 1H), 8.11 (t, J=7 Hz, 1H); MS (DCI-NH₃) m/z 376 (M+H)⁺.

### EXAMPLE 36

### (2E)-N-(4-hydroxybutyl)-3-{2-[(4-methylphenyl)thio]phenyl}acrylamide

### EXAMPLE 36A

### (2E)-ethyl 3-{2-[(4-methylphenyl)thio]phenyl}acrylate

The product from Example 5A and triethyl phosphonoacetate were processed as described in Example 1A to provide the title compound.

### EXAMPLE 36B

### (2E)-3-{2-[(4-methylphenyl)thio]phenyl}acrylic acid

The product from Example 36A was processed as described in Example 9A to provide the title compound.

### EXAMPLE 36C

### (2E)-N-(4-hydroxybutyl)-3-{2-[(4-methylphenyl)thio]phenyl}acrylamide

The product from Example 36B and 4-amino-1-butanol were processed as described in Example 1D to provide the title compound. ¹H NMR (300 MHz, DMSO-d₆) δ 1.44 (m, 4H), 2.29 (s, 3H), 3.16 (q, J=7 Hz, 2H), 3.42 (q, J=7 Hz, 2H), 4.39 (t, J=7 Hz, 1H), 6.60 (d, J=15 Hz, 1H), 7.20 (m, 5H), 7.35 (m, 2H), 7.66 (m, 1H), 7.86 (d, J=15 Hz, 1H), 8.11 (t, J=7 Hz, 1H); MS (DCI-NH₃) m/z 342 (M+H)⁺. FAB HRMS m/z for C₂₀H₂₄NO₂S (M+H)⁺: calcd 342.1528, observed 342.1537.

### EXAMPLE 37

### (2E)-3-{2-[(4-chlorophenyl)thio]phenyl}-N-(2-hydroxypropyl)acrylamide

The product from Example 9A and 1-amino-2-propanol were processed as described in Example 15A to provide the title compound. ¹H NMR (300 MHz, DMSO-d₆) δ 1.00 (d, J=7 Hz, 3H), 3.10 (m, 2H), 3.68 (m, 1H), 4.78 (br s, 1H), 6.74 (d, J=15 Hz, 1H), 7.19 (d, J=9 Hz, 2H), 7.42 (m, 5H), 7.73 (d, J=4 Hz, 1H), 7.82 (d, J=15 Hz, 1H), 8.12 (m, 1H); MS (ESI APCI+) m/z 348 (M+H)⁺.

### EXAMPLE 38

### (2E)-3-{2-[(4-chlorophenyl)thio]phenyl}-N-(3-hydroxybutyl)acrylamide

The product from Example 9A and 4-amino-2-butanol were processed as described in Example 15A to provide the title compound. ¹H NMR (300 MHz, DMSO-d₆) δ 1.02 (d, J=7 Hz, 3H), 1.50 (m, 2H), 3.12 (m, 2H), 3.63 (m, 1H), 4.60 (br s, 1H), 6.60 (d, J=15 Hz, 1H), 7.19 (d, J=9 Hz, 2H), 7.42 (m, 5H), 7.73 (d, J=4 Hz, 1H), 7.82 (d, J=15 Hz, 1H), 8.12 (m, 1H); MS (ESI APCI+) m/z 362 (M+H)⁺.

### EXAMPLE 39

### (2E)-3-{2-[(4-chlorophenyl)thio]phenyl}-N-(2-hydroxybutyl)acrylamide

The product from Example 9A and 1-amino-2-butanol were processed as described in Example 15A to provide the title compound. ¹H NMR (300 MHz, DMSO-d₆) δ 0.85 (t, J=7 Hz, 3H), 1.35-1.50 (m, 2H), 3.08 (m, 1H), 3.12 (m, 1H), 3.42 (m, 1H), 4.72 (br s, 1H), 6.72 (d, J=15 Hz, 1H), 7.19 (d, J=9 Hz, 2H), 7.42 (m, 5H), 7.73 (d, J=4 Hz, 1H), 7.82 (d, J=15 Hz, 1H), 8.12 (m, 1H); MS (ESI APCI+) m/z 362 (M+H)⁺.

### EXAMPLE 40

### (2E)-3-{2-[(4-chlorophenyl)thio]phenyl}-N-(2-hydroxycyclohexyl)acrylamide

The product from Example 9A and 2-aminocyclohexanol were processed as described in Example 15A to provide the title compound. ¹H NMR(300 MHz, DMSO-d₆) δ 1.20 (m, 4H), 1.60 (m, 2H), 1.82 (m, 2H), 3.22 (m, 1H), 3.50 (m, 1H), 4.05 (m, 1H), 4.60 (br s, 1H), 6.62 (d, J=15 Hz, 1H), 7.19 (d, J=9 Hz, 2H), 7.42 (m, 5H), 7.73 (d, J=4 Hz, 1H), 7.82 (d, J=15 Hz, 1H), 7.98 (m, 1H); MS (ESI APCI+) m/z 388 (M+H)⁺.

### EXAMPLE 41

### (2E)-3-{2-[(4-chlorophenyl)thio]-6-fluorophenyl}-N-(4-hydroxybutyl)acrylamide

### EXAMPLE 41A

### 2-[(4-Chlorophenyl)thio]-6-fluorobenzaldehyde

2,6-Difluorobenzaldehyde (2.3 g, 20 mmol), 4-chlorothiophenol (2.88 g, 20 mmol), and K₂CO₃ (4.14 g, 30 mmol) were combined in DMF (50 mL) and stirred at 50° C. for 16 hours. The mixture was poured into water (100 mL) and extracted with ethyl acetate. The acetate layer was washed with water, brine, dried over anhydrous MgSO₄, filtered, and the filtrate was concentrated under reduced pressure. The residue was purified by column chromatography to provide the title compound. MS (DCI/NH₃) m/z 284 (M+NH₄)⁺.

### EXAMPLE 41B

### (2E)-ethyl 3-{2-[(4-chlorophenyl)thio]-6-fluorophenyl}acrylate

The product from Example 41A and triethyl phosphonoacetate were processed as described in Example 1A to provide the title compound.

### EXAMPLE 41C

### (2E)-3-{2-[(4-chlorophenyl)thio]-6-fluorophenyl}acrylic acid

The product from Example 41B was processed as described in Example 9A to provide the title compound.

### EXAMPLE 41D

### (2E)-3-{2-[(4-chlorophenyl)thio]-6-fluorophenyl}-N-(4-hydroxybutyl)acrylamide

The product from Example 41C and 4-amino-1-butanol were processed as described in Example 1D to provide the title compound. ¹H NMR (300 MHz, CD₃OD) δ 1.59 (m, 4H), 3.3 (m, 2H), 3.58 (m, 2H), 6.71 (d, J=15 Hz, 1H), 7.25 (m, 7H), 7.86 (d, J=15 Hz, 1H); MS (DCI+) m/z 380 (M+H)⁺. FAB HRMS m/z for C₁₉H₂₀NO₂ClFS (M+H)⁺: calcd 380.0887; found 380.0875.

### EXAMPLE 42

### (2E)-N-(4-hydroxycyclohexyl)-3-{2-[(4-methylphenyl)thio]phenyl}acrylamide

The product from Example 36B and 4-aminocyclohexanol were processed as described in Example 29D to provide the title compound. ¹H NMR (300 MHz, DMSO-d₆) δ 1.21 (m, 4H), 1.45 (m, 4H), 2.29 (s, 3H), 3.4 (m, 1H), 3.56 (m, 1H), 4.51 (d, J=4.5 Hz, 1H), 6.58 (d, J=15 Hz, 1H), 7.18 (m, 5H), 7.34 (m, 2H), 7.62 (m, 1H), 7.85 (d, J=15 Hz, 1H), 7.99 (d, J=9 Hz, 1H); MS (DCI) m/z 368 (M+H)⁺.

### EXAMPLE 43

### (2E)-N-(4-hydroxybutyl)-3-{2-[(4-methoxyphenyl)thio]phenyl}acrylamide

### EXAMPLE 43A

### 3-(2-bromophenyl)-N-(4-hydroxybutyl)acrylamide

(2E)-3-(2-Bromophenyl)acrylic acid (4.06 g, 17.9 mmol) in methylene chloride (60 mL) was treated in succession with 1-(3-dimethylaminopropyl)-3-ethylcarbodiimide hydrochloride (6.84 g, 35.8 mmol), 1-hydroxybenzotriazole hydrate (2.42 g, 17.9 mmol), and neat 4-amino-1-butanol (3.19 g, 35.8 mmol) and stirred for 16 hours. The mixture was diluted with ethyl acetate and quenched with water. The aqueous phase was separated and extracted with ethyl acetate. All the ethyl acetate layers were combined, washed twice with a 1N aqueous HCl solution, twice with a saturated aqueous NaHCO₃ solution, brine solution, dried over MgSO₄, filtered, and the filtrate was concentrated under reduced pressure to provide the title compound. MS (CI+) m/z 299 (M+H)⁺.

### EXAMPLE 43B

### (2E)-N-(4-hydroxybutyl)-3-{2,[(4-methoxyphenyl)thio]phenyl}acrylamide

The product from Example 43A (400 mg, 1.34 mmol), 4-methoxybenzenethio] (166 mg, 1.34 mmol), copper pellets (4.2 mg,0.07 mmol), CuI (13.3 mg, 0.07 mmol), and K₂CO₃(555 mg, 4.02 mmol), were combined in N,N-dimethylformamide (5 mL) and heated at reflux for 16 hours. The mixture was allowed to cool to room temperature and diluted with ethyl acetate. The ethyl acetate layer was separated, washed twice with a 1N aqueous HCl solution, twice with a saturated aqueous NaHCO₃ solution, brine, dried over MgSO₄, filtered, and the filtrate concentrated under reduced pressure. The residue was purified by column chromatography (silica gel, 10% MeOH/CH₂Cl₂) to provide the title compound. mp 134-136° C.; ¹H NMR (300 MHz, CDCl₃) δ 1.66 (m, 4H), 3.44 (m, 2H), 3.72 (t, J=6.0 Hz, 2H), 3.81 (s, 3H), 5.89 (bm, 1H), 6.33 (d, J=15.0 Hz, 1H), 6.88 (m, 2H), 7.08 (m, 1H), 7.18 (m, 2H), 7.34 (m, 2H), 7.50 (m, 1H), 8.07 (d, J=15.0 Hz, 1H); MS (APCI+) m/z 358 (M+H)⁺; FAB HRMS m/z for C₂₀H₂₄NO₃S (M+H)⁺: calcd 358.1477; found 358.1492.

### EXAMPLE 44

### (2E)-N-(4-hydroxycyclohexyl)-3-{5-methoxy-2-[(4-methylphenyl)thio]phenyl}acrylamide

### EXAMPLE 44A

### (2E)-ethyl 3-{5-methoxy-2-[(4-methylphenyl)thio]phenyl}acrylate

Triethyl phosphonoacetate and 5-methoxy-2-[(4-methylphenyl)thio]benzaldehyde were processed as described in Example 1A to provide the title compound.

### EXAMPLE 44B

### (2E)-3-{5-methoxy-2-[(4-methylphenyl)thio]phenyl}acrylic acid

The product from Example 44A was processed as described in Example 9A to provide the title compound.

### EXAMPLE 44C

### (2E)-N-(4-hydroxycyclohexyl)-3-{5-methoxy-2-[(4-methylphenyl)thio]phenyl}acrylamide

The product from Example 44B and 4-aminocyclohexanol were processed as described in Example 1D to provide the title compound. ¹H NMR (300 MHz, DMSO-d₆) δ 1.21 (m, 4H), 1.80 (m, 4H), 3.38 (m, 1H), 2.30 (s, 3H), 3.38 (m, 1H), 3.59 (m, 1H), 4.51 (d, J=4.5 Hz, 1H), 6.47 (d, J=15 Hz, 1H), 6.65 (d, 4.5 Hz, 1H). 6.96 (dd, J=3 Hz, J=9 Hz, 1H), 7.20 (s, 4H), 7.60 (d, J=9 Hz, 1H), 7.78 (d, J=15 Hz, 1H), 7.90 (d, J=9 Hz, 1H); MS (DCI) m/z 398 (M+H)⁺.

### EXAMPLE 45

### (2E)-3-{2-[(4-fluorophenyl)thio]phenyl}-N-(2-hydroxycyclohexyl)acrylamide

The product from Example 21A (59 mg, 0.2 mmol) in anhydrous THF (1.0 mL) was treated with LDA (2.0 M, 0.5 mL, 1 mmol) at room temperature. After 30 minutes, the mixture was treated with 2-[(4-fluorophenyl)thio]benzaldehyde (35 mg, 0.15 mmol) in anhydrous THF (0.75 mL) and shaken at room temperature for 1 hour. The mixture was treated with water (0.3 mL) of water, concentrated, and the residue was purified by preparative HPLC to provide the title compound. ¹H NMR (300 MHz, DMSO-d₆) 1.20 (m, 4H), 1.60 (m, 2H), 1.82 (m, 2H), 3.30 (m, 1H), 3.55 (m, 1H), 4.58 (s, 1H), 6.64 (d, J=15 Hz, 1H), 7.2-07.45 (m, 6H), 7.55 (d, J=4 Hz, 1H), 7.67 (d, J=4 Hz, 1H), 7.84 (d, J=15 Hz, 1H), 8.00 (d, J=4 Hz, 1H); MS (ESI APCI+) m/z 375 (M+H)⁺.

### EXAMPLE 46

### (2E)-N-(2-hydroxycyclohexyl)-3-[2-(phenylthio)phenyl]acrylamide

The product from Example 21A and 2-(phenylthio)benzaldehyde were processed as described in Example 45 to provide the title compound. ¹H NMR (300 MHz, DMSO-d₆) δ 1.20 (m, 4H), 1.60 (m, 2H), 1.83 (m, 2H), 3.32 (m, 1H), 3.56 (m, 1H), 4.57 (s, 1H), 6.65 (d, J=15 Hz, 1H), 7.20-7.47 (m, 8H), 7.74 (d, J=4 Hz, 1H), 7.84 (d, J=15 Hz, 1H), 7.98 (d, J=4 Hz, 1H); MS (ESI APCI+) m/z 354 (M+H)⁺.

### EXAMPLE 47

### (2E)-N-(2-hydroxycyclohexyl)-3-{2-[(4-methylphenyl)thio]phenyl}acrylamide

The product from Example 21A and the product from Example 5A were processed as described in Example 45 to provide the title compound. ¹H NMR (300 MHz, DMSO-d₆) δ 1.20 (m, 4H), 1.60 (m, 2H), 1.83 (m, 2H), 2.29 (s, 3H), 3.29 (m, 1H), 3.57 (m, 1H), 4.58 (s, 1H), 6.62 (d, J=15 Hz, 1H), 7.20 (m, 5), 7.40 (m, 2H), 7.62 (d, J=3 Hz, 1H), 7.84 (d, J=15 Hz, 1H), 7.99 (d, J=4 Hz, 1H); MS (ESI APCI+) m/z 368 (M+H)⁺.

### EXAMPLE 48

### (2E)-N-(2-hydroxycyclohexyl)-3-{2-[(3-methoxyphenyl)thio]phenyl}acrylamide

The product from Example 21A and 2-[(3-methoxyphenyl)thio]benzaldehyde were processed as described in Example 45 to provide the title compound. ¹H NMR (300 MHz, DMSO-d₆) δ 1.20 (m, 4H), 1.60 (m, 2H), 1.85 (m, 2H), 3.29 (m, 1H), 3.57 (m, 1H), 3.70 (s, 3H), 4.59 (s, 1H), 6.64 (d, J=15 Hz, 1H), 6.68 (m, 2H), 6.81 (m, 1H), 7.22-7.42 (m, 4H), 7.70 (d, J=5 Hz, 1H), 7.84 (d, J=15 Hz, 1H), 7.99 (d, J=4 Hz, 1H); MS (ESI APCI+) m/z 384 (M+H)⁺.

### EXAMPLE 49

### (2E)-N-(2-hydroxycyclohexyl)-3-{2-[(4-methoxyphenyl)thio]phenyl}acrylamide

The product from Example 21A and 2-[(4-methoxyphenyl)thio]benzaldehyde were processed as described in Example 45 to provide the title compound. ¹H NMR (300 MHz, DMSO-d₆) δ 1.21 (m, 4H), 1.61 (m, 2H), 1.86 (m, 2H), 3.26 (m, 1H), 3.56 (m, 1H), 3.75 (s, 3H), 4.59 (s, 1H), 6.64 (d, J=15 Hz, 1H), 7.00-7.37 (m, 6H), 7.50 (d, J=5 Hz, 1H), 7.60 ((d, J=5 Hz, 1H), 7.82 (d, J=15 Hz, 1H), 7.99 (d, J=4 Hz, 1H); MS (ESI APCI+) m/z 384 (M+H)⁺.

### EXAMPLE 50

### (2E)-N-(4-hydroxycyclohexyl)-3-{2-[(3-methoxyphenyl)thio]phenyl}acrylamide

The product from Example 15A and 2-[(3-methoxyphenyl)thio]benzaldehyde were processed as described in Example 45 to provide the title compound. ¹H NMR (500 MHz, DMSO-d₆) 1.20 (m, 4H), 1.80 (m, 4H), 3.20 (m, 1H), 3.38 (m, 1H), 3.70 (s, 3H), 4.55 (br., 1H), 6.60 (d, J=15 Hz, 1H), 6.72 (m, 2H), 6.82 (d, J=3 Hz, 1H), 7.22 (m, 1H), 7.42 (m, 3H), 6.68 (d, J=3 Hz, 1H), 7.83 (d, J=15 Hz, 1H), 8.00 (d, J=3 Hz, 1H); MS (ESI APCI+) m/z 384 (M+H)⁺.

### EXAMPLE 51

### (2E)-N-(4-hydroxycyclohexyl)-3-{2-[(4-methoxyphenyl)thio]phenyl}acrylamide

The product from Example 15A and 2-[(4-methoxyphenyl)thio]benzaldehyde were processed as described in Example 45 to provide the title compound. ¹H NMR (500 MHz, DMSO-d₆) 1.22 (m, 4H), 1.80 (m, 4H), 3.20 (m, 1H), 3.40 (m, 1H), 3.78 (s, 3H), 4.55 (br., 1H), 6.58 (d, J=15 Hz, 1H), 7.00 (m, 3H), 7.30 (m, 2H), 7.38 (m, 2H), 7.58 (m, 1H), 7.86 (d, J=15 Hz, 1H), 8.00 (d, J=3 Hz, 1H); MS (ESI APCI+) m/z 384 (M+H)⁺.

### EXAMPLE 52

### (2E)-N-(4-hydroxycyclohexyl)-3-{2-[(3-methylphenyl)thio]phenyl}acrylamide

The product from Example 15A and 2-[(3-methylphenyl)thio]benzaldehyde were processed as described in Example 45 to provide the title compound. ¹H NMR (500 MHz, DMSO-d₆) 1.22 (m, 4H), 1.80 (m, 4H), 2.25 (s, 3H), 3.20 (m, 1H), 3.38 (m, 1H), 4.58 (br., 1H), 6.60 (d, J=15 Hz, 1H), 7.00 (d, J=3 Hz, 1H), 7.10 (d, J=3 Hz, 1H), 7.38 (m, 5H), 7.63 (d, J=3 Hz, 1H), 7.92 (d, J=15 Hz, 1H), 8.00 (d, J=3 Hz, 1H); MS (ESI APCI+) m/z 368 (M+H)⁺.

### EXAMPLE 53

### (2E)-3-{2-[(2,4-dimethylphenyl)thio]phenyl}-N-(5-hydroxy-1,5-dimethylhexyl)acrylamide

### EXAMPLE 53A

### 2-[(2,4-dimethylphenyl)thio]benzaldehyde

2-Nitrobenzaldehyde (1.51 g, 10 mmol), 2,4-dimethylthiophenol (1.66 g, 12 mmol), and K₂CO₃ (2.76 g 20 mmol) were combined in DMF (40 mL) and stirred at 50° C. for 16 hours. The mixture was allowed to cool to room temperature, poured into water (100 mL), and extracted with ethyl acetate. The acetate layer was washed with water, brine, dried over anhydrous MgSO₄, fitlered, and the filtrate concentrated under reduced pressure. The residue was purified by column chromatography to provide the title compound. MS (DCI/NH₃) m/z 243 (M+H)⁺, 260 (M+NH₄)⁺.

### EXAMPLE 53B

### (2E)-ethyl 3-{2-[(2,4-dimethylphenyl)thio]phenyl}acrylate

Triethyl phosphonoacetate and the product from Example 53A were processed as described in Example 1A to provide the title compound.

### EXAMPLE 53C

### (2E)-3-{2-[(2,4-dimethylphenyl)thio]phenyl}acrylic acid

The product from Example 53B was processed as described in Example 9A to provide the title compound.

### EXAMPLE 53D

### (2E)-3-{2-[(2,4-dimethylphenyl)thio]phenyl}-N-(5-hydroxy-1,5-dimethylhexyl)acrylamide

The product from Example 53C and 6-amino-2-methyl-2-heptanol were processed as described in Example 1D to provide the title compound. ¹H NMR (500 MHz, DMSO-d₆) δ 1.08 (s+d, 9H), 1.32 (m, 6H), 2.28 (2s, 6H), 3.85 (in, 1H), 4.06 (s, 1H), 6.60 (d, J=15 Hz, 1H), 6.90 (m, 1H), 7.03 (m, 1H), 7.10 (d, J=9 Hz, 1H), 7.18 (m, 1H), 7.29 (m, 2H), 7.60 (m, 1H), 7.82 (d, J=15 Hz, 1H), 7.98 (d, J=8 Hz, 1H); MS (DCI-NH₃) m/z 412 (M+H)⁺. Analysis calcd for C₂₅H₃₃NO₂S.0.75H₂O: C, 70.63; H, 8.18; N, 3.29. Found: C, 70.72; H, 8.20; N, 3.01.

### EXAMPLE 54

### (2E)-3-{2-[(4-chlorophenyl)thio]-5-fluorophenyl}-N-(4-hydroxybutyl)acrylamide

### EXAMPLE 54A

### (2E)-ethyl 3-{2-[(4-chlorophenyl)thio]-5-fluorophenyl}acrylate

Triethyl phosphonoacetate and 2-[(4-chlorophenyl)thio]-5-fluorobenzaldehyde were processed as described in Example 1A to provide the title compound.

### EXAMPLE 54B

### (2E)-3-{2-[(4-chlorophenyl)thio]-5-fluorophenyl}acrylic acid

The product from Example 54A was processed as described in Example 9A to provide the title compound.

### EXAMPLE 54C

### (2E)-3-{2-[(4-chlorophenyl)thio]-5-fluorophenyl}-N-(4-hydroxybutyl)acrylamide

The product from Example 54B and 4-amino-1-butanol were processed as described in Example 1D to provide the title compound. ¹H NMR (300 MHz, DMSO-d₆) δ 1.44 (m, 4H), 3.14 (q, J=7 Hz, 2H), 3.40 (q, J=7 Hz, 2H), 4.39 (t, J=7 Hz, 1H), 6.63 (d, J=15 Hz, 1H), 7.13 (m, 2H), 7.36 (m, 3H), 7.54 (m, 2H), 7.78 (d, J=15 Hz, 1H), 8.11 (t, J=7 Hz, 1H); MS (DCI-NH₃) m/z 380 (M+H)⁺. Analysis calcd for C₁₉H₁₉ClFNO₂S: C, 60.07; H, 5.04; N, 3.69. Found: C, 59.85; H, 4.99; N, 3.49.

### EXAMPLE 55

### (2E)-N-(4-hydroxycyclohexyl)-3-{2-[(3-methylbutyl)thio]phenyl}acrylamide

### EXAMPLE 55A

### 2-[(3-methylbutyl)thio]benzaldehyde

2-Fluorobenzaldehyde (571 mg, 4.6 mmol) in 10 ml DMF was treated in succession with solid potassium carbonate (700 mg, 5 mmol) and 3-methyl-1-butanethiol (600 mg, 5 mmol) in 5 ml of DMF at room temperature. The mixture was heated at 80° C. with stirring for 16 hours, allowed to cool to room temperature, filtrated, and the filtrate was concentrated under reduced pressure to provide the title compound which was used in next step without purification. ¹H NMR (500 MHz, DMSO-d₆) δ 0.92 (d, J=4 Hz, 6H), 1.50 (m, 2H), 1.75 (m, 1H), 3.00 (t, J=4 Hz, 2H), 7.38 (t, J=3 Hz, 1H), 7.52 (d, J=4 Hz, 1H), 7.63 (t, J=3 Hz, 1H), 7.88 (d, J=4 Hz, 1H), 10.21 (s, 1H).

### EXAMPLE 55B

### (2E)-3-{2-[(3-methylbutyl)thio]phenyl}acrylic acid

(Diethoxyphosphoryl)acetic acid (1 g, 5.1 mmol) in anhydrous THF (30 ml) was treated with LDA (6.1 mL, 2M in THF) under nitrogen at room temperature. After stirring for 15 minutes, the mixture was treated with the product from Example 55A (957 mg, 4.6 mmol) in anhydrous THF (10 mL) and stirred for 1 hour at room temperature. The mixture was concentrated, diluted with water (70 mL), adjusted to pH=2 with 3N HCl, and extracted with dichloromethane (x2). The organics phases were combined, dried over MgSO₄, filtered, and the filtrate concentrated under reduced pressure to provide the title compound. ¹H NMR (500 MHz, DMSO-d₆) δ 0.88 (d, J=4 Hz, 6H), 1.48 (m, 2H), 1.70 (m, 1H), 2.96 (t, J=4 Hz, 2H), 6.56 (d, J=15 Hz, 1H), 7.28 (m, 1H), 7.32 (m, 1H), 7.53 (m, 1H), 7.65 (m, 1H), 7.86 (d, J=15 Hz, 1H), 9.23 (br. s, 1H).

### EXAMPLE 55C

### (2E)-N-(4-hydroxycyclohexyl)-3-{2-[(3-methylbutyl)thio]phenyl}acrylamide

The product from Example 55B and 4-aminocyclohexanol were processed as described in Example 15A to provide the title compound. ¹H NMR (500 MHz, DMSO-d₆) δ 0.85 (d, J=3 Hz, 6H), 1.21 (m, 4H), 1.45 (m, 2H), 1.70 (m, 1H), 1.82 (m, 4H), 2.98 (m, 2H), 3.40 (br s, 1H), 3.58 (br s, 1H), 4.52 (s, 1H), 6.52 (d, J=15 Hz, 1H), 7.25 (t, J=3 Hz, 1H), 7.38 (t, J=3 Hz, 1H), 7.45 (d, J=4 Hz, 1H), 7.55 (d, J=4 Hz, 1H), 7.80 (d, J=15 Hz, 1H), 7.98 (d, J=3 Hz, 1H); MS (ESI APCI+) m/z 348 (M+H)⁺.

### EXAMPLE 56

### (2E)-N-(2-hydroxycyclohexyl)-3-{2-[(3-methylbutyl)thio]phenyl}acrylamide

The product from Example 55B and 2-aminocyclohexanol were processed as described in Example 15A to provide the title compound. ¹H NMR (500 MHz, DMSO-d₆) δ 0.85 (d, J=3 Hz, 6H), 1.21 (m, 4H), 1.45 (m, 2H), 1.62 (m, 2H), 1.70 (m, 1H), 1.85 (m, 2H), 2.98 (m, 2H), 3.55 (br s, 1H), 6.60 (d, J=15 Hz, 1H), 7.25 (t, J=3 Hz, 1H), 7.38 (t, J=3 Hz, 1H), 7.45 (d, J=4 Hz, 1H), 7.55 (d, J=4 Hz, 1H), 7.80 (d, J=15 Hz, 1H), 7.95 (d, J=3 Hz, 1H); MS (ESI APCI+) m/z 348 (M+H)⁺.

### EXAMPLE 57

### (2E)-N-(3-hydroxypropyl)-3-{2-[(3-methylbutyl)thio]phenyl}acrylamide

The product from Example 55B and 3-amino-1-propanol were processed as described in Example 15A to provide the title compound. ¹H NMR (500 MHz, DMSO-d₆) δ 0.85 (d, J=3 Hz, 6H), 1.45 (m, 2H), 1.60 (m, 2H), 1.70 (m, 1H), 2.95 (m, 2H), 3.12 (m, 2H), 3.45 (m, 2H), 6.55 (d, J=15 Hz, 1H), 7.25 (t, J=3 Hz, 1H), 7.35 (t, J=3 Hz, 1H), 7.45 (d, J=4 Hz, 1H), 7.58 (d, J=4 Hz, 1H), 7.82 (d, J=15 Hz, 1H), 8.12 (br s, 1H); MS (ESI APCI+) m/z 308 (M+H)⁺.

### EXAMPLE 58

### (2E)-N-(4-hydroxybutyl)-3-{2-[(3-methylbutyl)thio]phenyl}acrylamide

The product from Example 55B and 4-amino-1-butanol were processed as described in Example 15A to provide the title compound. ¹H NMR (500 MHz, DMSO-d₆) δ 0.85 (d, J=3 Hz, 6H), 1.45 (m, 6H), 1.70 (m, 1H), 2.95 (m, 2H), 3.18 (m, 2H), 3.42 (m, 2H), 6.52 (d, J=15 Hz, 1H), 7.25 (t, J=3 Hz, 1H), 7.38 (t, J=3 Hz, 1H), 7.45 (d, J=4 Hz, 1H), 7.58 (d, J=4 Hz, 1H), 7.82 (d, J=15 Hz, 1H), 8.10 (br s, 1H); MS (ESI APCI+) m/z 322 (M+H)⁺.

### EXAMPLE 59

### (2E)-N-(5-hydroxypentyl)-3-{2-[(3-methylbutyl)thio]phenyl}acrylamide

The product from Example 55B and 5-amino-1-pentanol were processed as described in Example 15A to provide the title compound. ¹H NMR (500 MHz, DMSO-d₆) δ 0.85 (d, J=3 Hz, 6H), 1.35 (m, 2H), 1.45 (m, 6H), 1.70 (m, 1H), 2.95 (m, 2H), 3.18 (m, 2H), 3.40 (m, 2H), 6.52 (d, J=15 Hz, 1H), 7.25 (t, J=3 Hz, 1H), 7.38 (t, J=3 Hz, 1H), 7.45 (d, J=4 Hz, 1H), 7.58 (d, J=4 Hz, 1H), 7.82 (d, J=15 Hz, 1H), 8.10(br s, 1H); MS (ESI APCI+) m/z 336 (M+H)⁺.

### EXAMPLE 60

### 1-((2E)-3-{2-[(3-methylbutyl)thio]phenyl}-2-propenoyl)-4-piperidinol

The product from Example 55B and 4-piperidinol were processed as described in Example 15A to provide the title compound. ¹H NMR (500 MHz, DMSO-d₆) δ 0.85 (d, J=3 Hz, 6H), 1.35 (m, 2H), 1.45 (m, 2H), 1.70 (m, 1H), 1.78 (m, 2H), 2.95 (m, 2H), 3.18 (m, 2H), 3.75 (m, 1H), 3.98 (m, 2H), 7.18 (d, J=15 Hz, 1H), 7.25 (t, J=3 Hz, 1H), 7.35 (t, J=3 Hz, 1H), 7.45 (d, J=4 Hz, 1H), 7.82 (d, J=4 Hz, 1H), 7.92 (d, J=15 Hz, 1H); MS (ESI APCI+) m/z 334 (M+H)⁺.

### EXAMPLE 61

### 1-((2E)-3-{2-[(3-methylbutyl)thio]phenyl}-2-propenoyl)-3-piperidinol

The product from Example 55B and 3-piperidinol were processed as described in Example 15A to provide the title compound. ¹H NMR (500 MHz, DMSO-d₆) δ 0.85 (d, J=3 Hz, 6H), 1.38 (m, 1H), 1.45 (m, 3H), 1.70 (m, 2H), 1.82 (m, 1H), 2.95 (m, 2H), 2.98 (m, 1H), 3.58 (m, 1H), 3.75 (m, 1H), 4.10 (m, 1H), 4.82 (m, 1H), 7.18 (m, 1H), 7.25 (t, J=3 Hz, 1H), 7.35 (t, J=3 Hz, 1H), 7.45 (m, 1H), 7.82 (m, 1H), 7.90(m, 1H); MS (ESI APCI+) m/z 334 (M+H)⁺.

### EXAMPLE 62

### 2-[1-((2E)-3-{2-[(3-methylbutyl)thio]phenyl}-2-propenoyl)-4-piperidinyl]ethanol

The product from Example 55B and 2-(4-piperidinyl)ethanol were processed as described in Example 15A to provide the title compound. ¹H NMR (500 MHz, DMSO-d₆) 0.85 (d, J=3 Hz, 6H), 1.45 (m, 2H), 1.70 (m, 1H), 2.95 (m, 2H), 3.12 (m, 4H), 3.22 (m, 2H), 3.55 (m, 2H), 3.78 (m, 2H), 4.50 (m, 2H), 5.38 (br s, 1H), 7.20 (d, J=15 Hz, 1H), 7.25 (t, J=3 Hz, 1H), 7.38 (t, J=3 Hz, 1H), 7.45 (d, J=4 Hz, 1H), 7.82 (d, J=4 Hz, 1H), 8.00 (d, J=15 Hz, 1H), 9.78 (br s, 1H); MS (ESI APCI+) m/z 363 (M+H)⁺.

### EXAMPLE 63

### 1-((2E)-3-{2-[(3-methylbutyl)thio]phenyl}-2-propenoyl)-4-piperidinecarboxamide

The product from Example 55B and 4-piperidinecarboxamide were processed as described in Example 15A to provide the title compound. ¹H NMR (500 MHz, DMSO-d₆) δ 0.85 (d, J=3 Hz, 6H), 1.45 (m, 4H), 1.70 (m, 1H), 1.78 (m, 3H), 2.38 (m, 1H), 2.72 (m, 1H), 2.95 (m, 2H), 3.08 (m, 1H), 4.25 (m, 1H), 4.42 (m, 1H), 6.78 (s, 1H), 7.18 (d, J=15 Hz, 1H), 7.25 (t, J=3 Hz, 1H), 7.38 (t, J=3 Hz, 1H), 7.45 (d, J=4 Hz, 1H), 7.82 (d, J=4 Hz, 1H), 7.92 (d, J=15 Hz, 1H); MS (ESI APCI+) m/z 361 (M+H)⁺.

### Guanylate Cyclase Assay

Sf9 cells were cultured in Sf-90011 medium supplemented with 10% (v/v) fetal calf serum at 28° C. to give a count of 6×105 cells/ml. The cells were then infected with a mixture of the virus stocks for the α1 and β1 subunits of guanylate cyclase at a multiplicity of infection of 0.1 pfu/cell. The cells were harvested 72 hours after infection and were pelleted by centrifugation. The resulting cell pellets were homogenized with Polytron homogenizer in 3 volumes of 20 mM Tris-HCl (pH 7.6) containing 1 mM EDTA, 5 mM dithiothreitol, 90 mM NaCl, 10% glycerol, and protease inhibitors mix (Roche Molecular Biochemicals). The homogenate was centrifuged at 50,000×g for 60 minutes and the supernatant was aliquotted and stored at −80° C.

Compounds of the present invention were dissolved in DMSO to make 100 mM and saved as stocks at −20° C. When compounds were tested, prepared 10-fold concentrated solutions with 10% DMSO. Then added 10 μl of 10-fold concentrated compound solutions to the reaction mixture of total 100 μl.

Guanylate cyclase activity was measured by the formation of cyclic GMP from GTP in a total volume of 100 μl. Guanylate cyclase-expressed Sf9 cell high-speed supernatant was used as an enzyme source for the assay. The reaction mixture containing 0.1 μl enzyme (Sf9 high-speed sup), compound solution, 50 mM Tris-HCl (pH 7.6), 4 mM MgCl₂, 0.5 mM IBMX, 15 mM creatine phosphate, 20 μg creatine phosphokinase, 0.1% BSA and 1 mM GTP was incubated at 37° C. for 15 minutes. After the reaction, 0.9 ml of 50 mM sodium acetate buffer (pH 5.8) was added followed by boiling for 3 minutes to stop the reaction. The solution was centrifuged at 3000 rpm for 15 minutes at room temperature and cyclic GMP in 20 μl of the resulting supernatant was measured according to the protocol of Amersham cyclic GMP enzymeimmunoassay system (purchased from Sigma, catalogue number RPN 226).

Compounds of the present invention were tested at 100 μM with and without 1 μM sodium nitro prusside (SNP). 1-Benzyl-3-hydroxymethyl-2-furylindazole (YC-1) was included as a standard for each assay. The data was normalized with the activity of 100 μM YC-1 (without SNP) as 100%. Mean basal efficacy, in Table 1, was expressed as a percentage of 100 μM YC-1 (without SNP).

The mean activation, in Table 1, was calculated based on the formula: (activity of compound of the present invention at 100 μM with 1 μM SNP)−(activity of compound of the present invention at 100 μM)/(activity of SNP at 1 μM)−(basal activity of sGC).

TABLE 1

| | Mean basal | | |
|---|---|---|---|
| | efficacy of | Mean efficacy of | |
| | compound alone | compound | |
| Example | at 100 μM, | combined with | Mean activation at |
| Number | [%] | 1 μM of SNP, [%] | 100 μM |
| YC-1 | 100 | 224 | 2.7 |
| 1 | 4 | 25 | 2.4 |
| 2 | 9 | 84 | 2.6 |
| 3 | 2 | 29 | 2.3 |
| 4 | 1 | 28 | 2.6 |
| 5 | 14 | 96 | 5.1 |
| 6 | 2 | 11 | 2.7 |
| 7 | 19 | 80 | 3.8 |
| 8 | 0 | 16 | 3.1 |
| 9 | 4 | 22 | 4.0 |
| 10 | 0 | 7 | 1.6 |
| 11 | 1 | 6 | 2.8 |
| 12 | 2 | 20 | 1.5 |
| 13 | 4 | 25 | 2.4 |
| 14 | 30 | 124 | 4.4 |
| 15 | 126 | 204 | 4.7 |
| 16 | 11 | 68 | 3.6 |
| 17 | 38 | 138 | 5.4 |
| 18 | 43 | 113 | 4.1 |
| 19 | 37 | 120 | 4.8 |
| 20 | 90 | 199 | 6.3 |
| 21 | 45 | 106 | 4.1 |
| 22 | 158 | 169 | 3.9 |
| 23 | 174 | 187 | 2.7 |
| 24 | 4 | 17 | 1.6 |
| 25 | 242 | 430 | 9.5 |
| 26 | 353 | 506 | 7.9 |
| 27 | 115 | 278 | 8.3 |
| 28 | 6 | 18 | 2.6 |
| 29 | 69 | 110 | 2.9 |
| 30 | 66 | 289 | 3.4 |
| 31 | 93 | 187 | 3.7 |
| 32 | 27 | 78 | 10.5 |
| 33 | 7 | 38 | 6.9 |
| 34 | 113 | 219 | 6.9 |
| 35 | 49 | 228 | 6.3 |
| 36 | 66 | 212 | 9.2 |
| 37 | 70 | 405 | 30.6 |
| 38 | 60 | 259 | 19.0 |
| 39 | 73 | 305 | 19.3 |
| 40 | 100 | 366 | 6.3 |
| 41 | 5 | 69 | 3.8 |
| 42 | 14 | 58 | 4.8 |
| 43 | 54 | 160 | 9.5 |
| 44 | 36 | 89 | 3.5 |
| 45 | 16 | 76 | 4.0 |
| 46 | 54 | 121 | 4.3 |
| 47 | 16 | 44 | 2.8 |
| 48 | 20 | 102 | 4.6 |
| 49 | 15 | 79 | 3.7 |
| 50 | 68 | 175 | 6.4 |
| 51 | 119 | 258 | 7.2 |
| 52 | 11 | 50 | 2.7 |
| 53 | 4 | 10 | 1.7 |
| 54 | 85 | 186 | 13.0 |
| 55 | 143 | 213 | 5.3 |
| 56 | 169 | 276 | 8.3 |
| 57 | 200 | 303 | 8.4 |
| 58 | 215 | 307 | 7.8 |
| 59 | 162 | 221 | 8.4 |
| 60 | 115 | 165 | 8.3 |
| 61 | 103 | 165 | 10.2 |
| 62 | 136 | 161 | 4.7 |
| 63 | 125 | 164 | 6.8 |

The data in Table 1 indicates that compounds of the present invention potentiate the activation of sGC by nitric oxide resulting in increased levels of cGMP. Activation of sGC by allosteric activators or non NO-donors potentiates the effect of NO released during sexual stimulation increasing cGMP. Therefore, compounds of the present invention are useful for sexual dysfunction in mammals including male erectile dysfunction.

Compounds of the present invention potentiate the activation of sGC by nitric oxide resulting in increased levels of cGMP and therefore compounds of the present invention are useful in combination with phosphodiesterase 5 inhibitors including, but not limited to, sildenafil or vardenafil as a method of treating sexual dysfunction in a mammal.

Compounds of the present invention potentiate the activation of sGC by nitric oxide resulting in increased levels of cGMP and therefore compounds of the present invention are useful in combination with an adrenergic receptor antagonist including, but not limited to, terazosin, prazosin or tamsulosin as a method of treating sexual dysfunction in a mammal.

Compounds of the present invention potentiate the activation of sGC by nitric oxide resulting in increased levels of cGMP and therefore compounds of the present invention are useful in combination with a dopamine agonist including, but not limited to, apomorphine as a method of treating sexual dysfunction in a mammal.

Compounds of the present invention potentiate the activation of sGC by nitric oxide resulting in increased levels of cGMP and therefore compounds of the present invention are useful in disorders associated with low levels of cGMP such as cardiovascular disease, atherosclerosis, angina pectoris, diastolic dysfunction, benign prostatic hyperplasia (BPH), incontinence, sexual dysfunction, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders as discussed in S. Moncada, E. A. Higgs, FASEB J., 9(13), 1319-1330 (1995); I. S. Severina, Biochemistry (Mosc), 63(7), 794-801 (1998); Y-C. Lee, E. Martin, F. Murad, PNAS, 97(20), 10763-10768 (2000); A. J. Hobbs, TIPS, 18, 484-491 (1997); F. Murad, Adv. Pharmacol., 26, 1-335 (1994); and J. W. Denninger, M. A. Marletta, Biochim. Biophys. Acta, 1411, 334-350 (1999).

The present invention also provides pharmaceutical compositions that comprise compounds of the present invention. The pharmaceutical compositions comprise compounds of the present invention formulated together with one or more nontoxic pharmaceutically acceptable carriers. The pharmaceutical compositions can be specially formulated for oral administration in solid or liquid form, for parenteral injection or for rectal administration.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, prcpylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of this invention with suitable non-irritating carriers or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound of this invention include powders, sprays, ointments and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated.

When used in the above or other treatments, a therapeutically effective amount of one of the compounds of the present invention can be employed in pure form or, where such forms exist, in pharmaceutically acceptable salt, ester or prodrug form. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The compounds of the present invention can be used in the form of pharmaceutically acceptable salts derived from inorganic or organic acids. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio.

Pharmaceutically acceptable salts are well-known in the art. For example, S. M. Berge et al. describe pharmaceutically acceptable salts in detail in (J. Pharmaceutical Sciences, 1977, 66: 1 et seq). The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts can be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to compounds of formula (I), for example, by hydrolysis in blood.

The present invention contemplates compounds of formula (I) formed by synthetic means or formed by in vivo biotransformation.

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The total daily dose of the compounds of this invention administered to a human may range from about 0.01 to about 50 mg/kg/day. A more preferred total daily dose may range from about 0.5 mg/kg/day to about 5 mg/kg/day. If desired, the effective daily dose can be divided into multiple doses for purposes of administration; consequently, single dose compositions may contain such amounts or submultiples thereof to make up the daily dose.

What is claimed is:

1. A compound of formula (I) 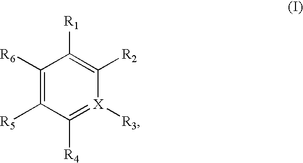
or a pharmaceutically acceptable salt, ester, amide or prodrug thereof, wherein
X is selected from the group consisting of C and N;
R₁ is selected from the group consisting of (NR7R8)carbonylalkyl-, and (NR7R8)carbonylalkenyl-;
R₂ is selected from the group consisting of alkoxy, alkylthio, aryloxy, arylthio, cycloalkyloxy, and cycloalkylthio;
provided that when X is C and R₂ is arylthio, then R₁ is (NR7R8)carbonylalkyl-;
R₃ is absent or selected from the group consisting of hydrogen, alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, and (NR9R10)carbonyl-;
R₄, R₅, and R₆ are independently selected from the group consisting of hydrogen, alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, and (NR9R10)carbonyl-;
R₇ and R₈ are independently selected from the group consisting of hydrogen, alkyl, aryl, arylalkyl, cycloalkyl, cycloalkylalkyl, heterocycle, heterocyclealkyl, hydroxyalkyl, and (NHR11)alkyl-; or
R₇ and R₈ taken together with the nitrogen atom to which they are attached, together form a heterocycle selected from the group consisting of 1-azetidinyl, 1-azepanyl, 1-aziridinyl, 4-morpholinyl, 1-piperazinyl, 1-piperidinyl, 1-pyrrolidinyl, and 1,1-dioxido-4-thiomorpholinyl, wherein the heterocycle is substituted with 0, 1, 2, 3, or 4 substituents selected from the group consisting of alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, and (NR9R10)carbonyl-;
R₉ and R₁₀ are independently selected from the group consisting of hydrogen and alkyl; and
R₁₁ is selected from the group consisting of hydrogen, alkoxy, alkyl, and alkylsulfonyl.

2. A compound according to claim 1 wherein
X is C;
R₁ is (NR7R8)carbonylalkenyl-; and
R₂ is alkylthio.

3. A compound according to claim 1 wherein
X is C;
R₁ is (NR7R8)carbonylalkenyl-;
R₂ is alkylthio;
R₃, R₄, R₅, and R₆ are independently selected from the group consisting of hydrogen, alkoxy, alkyl, and halogen;
R₇ is cycloalkyl substituted with 0, 1, or 2 substituents selected from the group consisting of alkyl and hydroxy; and
R₈ is selected from the group consisting of hydrogen and alkyl.

4. A compound according to claim 3 selected from the group consisting of
(2E)-N-(2-hydroxycyclohexyl)-3-[2-(isobutylthio)phenyl]acrylamide;
(2E)-N-(4-hydroxycyclohexyl)-3-[2-(isopropylthio)phenyl]acrylamide;
(2E)-N-(4-hydroxycyclohexyl)-3-[2-(pentylthio)phenyl]acrylamide; and
(2E)-N-(2-hydroxycyclohexyl)-3-{2-[(3-methylbutyl)thio]phenyl}acrylamide.

5. A compound according to claim 3 that is (2E)-N-(4-hydroxycyclohexyl)-3-{2-[(3-methylbutyl)thio]phenyl}acrylamide.

6. A compound according to claim 1 wherein
X is C;
R₁ is (NR7R8)carbonylalkenyl-;
R₂ is alkylthio;
R₃, R₄, R₅, and R₆ are independently selected from the group consisting of hydrogen, alkoxy, alkyl, and halogen;
R₇ is hydroxyalkyl; and
R₈ is selected from the group consisting of hydrogen and alkyl.

7. A compound according to claim 6 selected from the group consisting of
(2E)-N-(3-hydroxypropyl)-3-{2-[(3-methylbutyl)thio]phenyl}acrylamide;
(2E)-N-(4-hydroxybutyl)-3-{2-[(3-methylbutyl)thio]phenyl}acrylamide; and
(2E)-N-(5-hydroxypentyl)-3-{2-[(3-methylbutyl)thio]phenyl}acrylamide.

8. A compound according to claim 1 wherein
X is C;
R₁ is (NR7R8)carbonylalkenyl-;
R₂ is alkylthio; and
R₇ and R₈ taken together with the nitrogen atom to which they are attached, together form a heterocycle selected from the group consisting of 1-azetidinyl, 1-azepanyl, 1-aziridinyl, 4-morpholinyl, 1-piperidinyl, 1-pyrrolidinyl, and 1,1-dioxido-4-thiomorpholinyl, wherein the heterocycle is substituted with 0, 1, 2, 3, or 4 substituents selected from the group consisting of alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₉R₁₀, and (NR9R10)carbonyl-.

9. A compound according to claim 1 wherein
X is C;
R₁ is (NR7R8)carbonylalkenyl-;
R₂ is alkylthio;
R₃, R₄, R₅, and R₆ are independently selected from the group consisting of hydrogen, alkoxy, alkyl, and halogen; and
R₇ and R₈ taken together with the nitrogen atom to which they are attached, together form piperidinyl substituted with 0, 1, or 2 substituents selected from the group consisting of alkyl, hydroxy, hydroxyalkyl, and (NR9R10)carbonyl-.

10. A compound according to claim 9 selected from the group consisting of
1-((2E)-3-{2-[(3-methylbutyl)thio]phenyl}-2-propenoyl)-4-piperidinol;
1-((2E)-3-{2-[(3-methylbutyl)thio]phenyl}-2-propenoyl)-3-piperidinol;
2-[1-((2E)-3-{2-[(3-methylbutyl)thio]phenyl}-2-propenoyl)-4-piperidinyl]ethanol; and
1-((2E)-3-{2-[(3-methylbutyl)thio]phenyl}-2-propenoyl)-4-piperidinecarboxamide.

11. A compound according to claim 1 wherein
X is C;
R₁ is (NR7R8)carbonylalkenyl-; and
R₂ is cycloalkylthio.

12. A compound according to claim 1 wherein
X is C;
R₁ is (NR7R8)carbonylalkenyl-;
R₂ is cycloalkylthio;
R₃, R₄, R₅, and R₆ are independently selected from the group consisting of hydrogen, alkoxy, alkyl, and halogen;
R₇ is cycloalkyl substituted with 0, 1, or 2 substituents selected from the group consisting of alkyl and hydroxy; and
R₈ is selected from the group consisting of hydrogen and alkyl.

13. A compound according to claim 12 selected from the group consisting of
(2E)-3-[2-(cyclopentylthio)phenyl]-N-(2-hydroxycyclohexyl)acrylamide;
(2E)-3-[2-(cyclohexylthio)phenyl]-N-(2-hydroxycyclohexyl)acrylamide;
(2E)-3-[2-(cyclopentylthio)phenyl]-N-(4-hydroxycyclohexyl)acrylamide; and
(2E)-3-[2-(cyclohexylthio)phenyl]-N-(4-hydroxycyclohexyl)acrylamide.

14. A compound according to claim 1 wherein
X is C;
R₁ is (NR7R8)carbonylalkyl-; and
R₂ is arylthio.

15. A compound according to claim 1 wherein
X is C;
R₁ is (NR7R8)carbonylalkyl-;
R₂ is arylthio wherein the aryl of arylthio is phenyl substituted with 0, 1, or 2 substituents selected from the group consisting of alkoxy, alkyl and halogen;
R₃, R₄, R₅, and R₆ are independently selected from the group consisting of hydrogen, alkoxy, alkyl, and halogen;
R₇ is cycloalkyl substituted with 0, 1, or 2 substituents selected from the group consisting of alkyl and hydroxy; and
R₈ is selected from the group consisting of hydrogen and alkyl.

16. A compound according to claim 15 that is 3-{2-[(4-chlorophenyl)thio]phenyl}-N-(4-hydroxycyclohexyl)propanamide.

17. A compound according to claim 1 wherein
X is C;
R₁ is (NR7R8)carbonylalkyl-;
R₂ is arylthio wherein the aryl of arylthio is phenyl substituted with 0, 1, or 2 substituents selected from the group consisting of alkoxy, alkyl and halogen;
R₃, R₄, R₅, and R₆ are independently selected from the group consisting of hydrogen, alkoxy, alkyl, and halogen;
R₇ is selected from the group consisting of hydroxyalkyl and (NHR11)alkyl-;
R₈ is selected from the group consisting of hydrogen and alkyl; and
R₁₁ is selected from the group consisting of hydrogen, alkoxy, alkyl, and alkylsulfonyl.

18. A compound according to claim 17 selected from the group consisting of
3-{2-[(4-chlorophenyl)thio]phenyl}-N-(4-hydroxybutyl)propanamide;
3-{2-[(4-chlorophenyl)thio]phenyl}-N-(5-hydroxy-1,5-dimethylhexyl)propanamide;
3-{2-[(4-chlorophenyl)thio]phenyl}-N-(5-hydroxypentyl)propanamide;
N-(4-hydroxybutyl)-3-{2-[(4-methylphenyl)thio]phenyl}propanamide;
3-{2-[(4-chlorophenyl)thio]phenyl}-N-{4-[(methylsulfonyl)amino]butyl}propanamide;
3-{2-[(4-chlorophenyl)thio]-3-fluorophenyl}-N-(4-hydroxybutyl)propanamide;
3-{2-[(4-chlorophenyl)thio]-5-fluorophenyl}-N-(4-hydroxybutyl)propanamide;
3-{2-[(4-chlorophenyl)thio]phenyl}-N-(4-hydroxypentyl)propanamide;
3-{2-[(4-chlorophenyl)thio]phenyl}-N-[4-(methoxyamino)butyl]propanamide;
3-{2-[(4-chlorophenyl)thio]phenyl}-N-[4-(methylamino)butyl]propanamide; and
3-{2-[(4-chlorophenyl)thio]phenyl}-N-[5-(methylamino)pentyl]propanamide.

19. A compound according to claim 1 wherein
X is C;
R₁ is (NR7R8)carbonylalkyl-;
R₂ is arylthio wherein the aryl of arylthio is phenyl substituted with 0, 1, or 2 substituents selected from the group consisting of alkoxy, alkyl and halogen;
R₃, R₄, R₅, and R₆ are independently selected from the group consisting of hydrogen, alkoxy, alkyl, and halogen;
R₇ is heterocycle; and
R₈ is selected from the group consisting of hydrogen and alkyl.

20. A compound according to claim 1 wherein
X is C;
R₁ is (NR7R8)carbonylalkyl-;
R₂ is arylthio wherein the aryl of arylthio is phenyl substituted with 0, 1, or 2 substituents selected from the group consisting of alkoxy, alkyl and halogen;
R₃, R₄, R₅, and R₆ are independently selected from the group consisting of hydrogen, alkoxy, alkyl, and halogen;
R₇ is heterocycle wherein the heterocycle is piperidinyl substituted with 0, 1, or 2 substituents selected from the group consisting of alkoxycarbonyl and alkyl; and
R₈ is selected from the group consisting of hydrogen and alkyl.

21. A compound according to claim 20 that is 3-{2-[(4-chlorophenyl)thio]phenyl}-N-methyl-N-(1-methyl-4-piperidinyl)butanamide.

22. A compound according to claim 1 wherein
X is C;
R₁ is (NR7R8)carbonylalkyl-;
R₂ is arylthio wherein the aryl of arylthio is phenyl substituted with 0, 1, or 2 substituents selected from the group consisting of alkoxy, alkyl and halogen;
R₃, R₄, R₅, and R₆ are independently selected from the group consisting of hydrogen, alkoxy, alkyl, and halogen;
R₇ is heterocyclealkyl; and
R₈ is selected from the group consisting of hydrogen and alkyl.

23. A compound according to claim 1 wherein
X is C;
R₁ is (NR7R8)carbonylalkyl-;
R₂ is arylthio wherein the aryl of arylthio is phenyl substituted with 0, 1, or 2 substituents selected from the group consisting of alkoxy, alkyl and halogen;
R₃, R₄, R₅, and R₆ are independently selected from the group consisting of hydrogen, alkoxy, alkyl, and halogen;
R₇ is heterocyclealkyl wherein the heterocycle of heterocyclealkyl is pyridinyl substituted with 0, 1, or 2 substituents selected from the group consisting of alkoxy, alkyl, and halogen; and
R₈ is selected from the group consisting of hydrogen and alkyl.

24. A compound according to claim 23 that is 3-{2-[(4-chlorophenyl)thio]phenyl}-N-[2-(4-pyridinyl)ethyl]butanamide.

25. A compound according to claim 1 wherein
X is N;
R₁ is (NR7R8)carbonylalkenyl-; and
R₂ is arylthio.

26. A compound according to claim 1 wherein
X is N;
R₁ is (NR7R8)carbonylalkenyl-;
R₂ is arylthio wherein the aryl of arylthio is phenyl substituted with 0, 1, or 2 substituents selected from the group consisting of alkoxy, alkyl and halogen;
R₃ is absent;
R₄, R₅, and R₆ are independently selected from the group consisting of hydrogen, alkoxy, alkyl, and halogen;
R₇ is cycloalkyl substituted with 0, 1, or 2 substituents selected from the group consisting of alkyl and hydroxy; and
R₈ is selected from the group consisting of hydrogen and alkyl.

27. A compound according to claim 26 selected from the group consisting of
(2E)-N-(4-hydroxycyclohexyl)-3-{2-[(4-methylphenyl)thio]-3-pyridinyl}acrylamide;
(2E)-3-{2-[(4-chlorophenyl)thio]-3-pyridinyl}-N-(4-hydroxycyclohexyl)acrylamide;
(2E)-3-{2-[(2,4-dichlorophenyl)thio]-3-pyridinyl}-N-(4-hydroxycyclohexyl)acrylamide;
(2E)-3-{2-[(4-bromophenyl)thio]-3-pyridinyl}-N-(4-hydroxycyclohexyl)acrylamide;
(2E)-N-(4-hydroxycyclohexyl)-3-[2-(phenylthio)-3-pyridinyl]acrylamide;
(2E)-3-{2-[(2-chlorophenyl)thio]-3-pyridinyl}-N-(4-hydroxycyclohexyl)acrylamide; and
(2E)-3-{2-[(4-chlorophenyl)thio]-6-methyl-3-pyridinyl}-N-(4-hydroxycyclohexyl)acrylamide.

28. A method of treating a disorder ameliorated by increasing cGMP levels in a mammal comprising administering to the mammal a therapeutically effective amount of a compound of formula (I).

29. The method according to claim 28 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

30. The method according to claim 28 wherein the disorder is sexual dysfunction.

31. The method according to claim 28 wherein the disorder is male erectile dysfunction.

32. A method of treating a disorder ameliorated by increasing cGMP levels in a mammal comprising administering to the mammal a therapeutically effective amount of a compound of formula (I) in combination with a pharmaceutically acceptable carrier.

33. The method according to claim 32 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

34. The method according to claim 32 wherein the disorder is sexual dysfunction.

35. The method according to claim 32 wherein the disorder is male erectile dysfunction.

36. A method of treating a disorder ameliorated by increasing cGMP levels in a mammal comprising administering to the mammal a therapeutically effective amount of a compound of formula (I) in combination with a phosphodiesterase 5 inhibitor.

37. The method according to claim 36 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

38. The method according to claim 36 wherein the disorder is sexual dysfunction.

39. The method according to claim 36 wherein the disorder is male erectile dysfunction.

40. A method of treating a disorder ameliorated by increasing cGMP levels in a mammal comprising administering to the mammal a therapeutically effective amount of a compound of formula (I) in combination with an adrenergic receptor antagonist.

41. The method according to claim 40 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

42. The method according to claim 40 wherein the disorder is sexual dysfunction.

43. The method according to claim 40 wherein the disorder is male erectile dysfunction.

44. A method of treating a disorder ameliorated by increasing cGMP levels in a mammal comprising administering to the mammal a therapeutically effective amount of a compound of formula (I) in combination with a dopamine receptor agonist.

45. The method according to claim 44 wherein the disorder is selected from the group consisting of cardiovascular disease, atherosclerosis, angina pectoris, diastolic dysfunction, benign prostatic hyperplasia (BPH), incontinence, endothelial dysfunction, trombosis, diabetes, liver cirhosis, cognitive disorders, Alzheimer's disease, anxiety, stress, depression, sleep disorders, migraine, cerebral ischemia, brain trauma, pain, memory and learning disorders.

46. The method according to claim 44 wherein the disorder is sexual dysfunction.

47. The method according to claim 44 wherein the disorder is male erectile dysfunction.

48. A method of treating sexual dysfunction in a mammal comprising administering to said mammal in need of such treatment a therapeutically effective amount of a compound of formula (II) 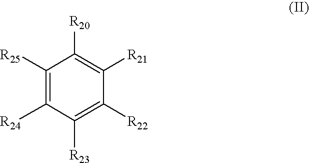
or a pharmaceutically acceptable salt, ester, amide, or prodrug thereof, wherein
R₂₀ is (NR₂₆R₂₇)carbonylalkenyl;
R₂₁ is arylthio wherein the aryl of arylthio is phenyl substituted with 0, 1, 2, 3, 4, or 5 substituents selected from the group consisting of alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₂₈R₂₉, and (NR₂₈R₂₉)carbonyl;
R₂₂, R₂₃, R₂₄, and R₂₅ are independently selected from the group consisting of hydrogen, alkenyl, alkoxy, alkoxycarbonyl, alkyl, alkylcarbonyl, alkylthio, carboxy, cyano, haloalkoxy, haloalkyl, halogen, hydroxy, hydroxyalkyl, mercapto, mercaptoalkyl, nitro, —NR₂₈R₂₉, and (NR₂₈R₂₉)carbonyl;
R₂₆ and R₂₇ are independently selected from the group consisting of hydrogen, alkyl, cycloalkyl, and hydroxyalkyl; and
R₂₈ and R₂₉ are independently selected from the group consisting of hydrogen and alkyl.

49. The method according to claim 48 wherein
R₂₁ is arylthio wherein the aryl of arylthio is phenyl substituted with 0, 1, 2, or 3 substituents selected from the group consisting of alkoxy, alkyl, and halogen;
R₂₂, R₂₃, R₂₄, and R₂₅ are independently selected from the group consisting of hydrogen, alkoxy, and halogen;
R₂₆ is cycloalkyl substituted with 0, 1, or 2 substituents selected from the group consisting of alkyl, hydroxy, and oxo; and
R₂₇ is selected from the group consisting of hydrogen and alkyl.

50. The method according to claim 48 selected from the group consisting of
(2E)-3-{2-[(4-chlorophenyl)thio]phenyl}-N-(4-hydroxycyclohexyl)acrylamide;
(2E)-3-{2-[(4-chlorophenyl)thio]phenyl}-N-(2-hydroxycyclohexyl)acrylamide;
(2E)-N-(4-hydroxycyclohexyl)-3-{2-[(4-methylphenyl)thio]phenyl}acrylamide;
(2E)-N-(4-hydroxycyclohexyl)-3-{5-methoxy-2-[(4-methylphenyl)thio]phenyl}acrylamide;
(2E)-3-{2-[(4-fluorophenyl)thio]phenyl}-N-(2-hydroxycyclohexyl)acrylamide;
(2E)-N-(2-hydroxycyclohexyl)-3-[2-(phenylthio)phenyl]acrylamide;
(2E)-N-(2-hydroxycyclohexyl)-3-{2-[(4-methylphenyl)thio]phenyl}acrylamide;
(2E)-N-(2-hydroxycyclohexyl)-3-{2-[(3-methoxyphenyl)thio]phenyl}acrylamide;
(2E)-N-(2-hydroxycyclohexyl)-3-{2-[(4-methoxyphenyl)thio]phenyl}acrylamide;
(2E)-N-(4-hydroxycyclohexyl)-3-{2-[(3-methoxyphenyl)thio]phenyl}acrylamide;
(2E)-N-(4-hydroxycyclohexyl)-3-{2-[(4-methoxyphenyl)thio]phenyl}acrylamide; and
(2E)-N-(4-hydroxycyclohexyl)-3-{2-[(3-methylphenyl)thio]phenyl}acrylamide.

51. The method according to claim 48 wherein
R₂₁ is arylthio wherein the aryl of arylthio is phenyl substituted with 0, 1, 2, or 3 subtituents selected from the group consisting of alkoxy, alkyl, and halogen;
R₂₂, R₂₃, R₂₄, and R₂₅ are independently selected from the group consisting of hydrogen, alkoxy, and halogen;
R₂₆ is selected from the group consisting of alkyl and hydroxyalkyl;
R₂₇ is selected from the group consisting of hydrogen and alkyl.

52. A compound according to claim 51 selected from the group consisting of
(2E)-3-{2-[(2,4-dichlorophenyl)thio]phenyl}-N-(4-hydroxybutyl)acrylamide;
(2E)-3-{2-[(4-chlorophenyl)thio]phenyl}-N-(5-hydroxy-1,5-dimethylhexyl)acrylamide;
(2E)-3-{2-[(4-chlorophenyl)thio]phenyl}-N-ethylacrylamide;
(2E)-N-butyl-3-{2-[(4-chlorophenyl)thio]phenyl}acrylamide;
(2E)-3-{2-[(4-chlorophenyl)thio]phenyl}-N-(4-hydroxybutyl)acrylamide;
(2E)-3-{2-[(4-chlorophenyl)thio]phenyl}-N-(5-hydroxypentyl)acrylamide;
(2E)-N-(4-hydroxybutyl)-3-{2-[(4-methylphenyl)thio]phenyl}acrylamide;
(2E)-3-{2-[(4-chlorophenyl)thio]phenyl}-N-(2-hydroxypropyl)acrylamide;
(2E)-3-{2-[(4-chlorophenyl)thio]phenyl}-N-(3-hydroxybutyl)acrylamide;
(2E)-3-{2-[(4-chlorophenyl)thio]phenyl}-N-(2-hydroxybutyl)acrylamide;
(2E)-3-{2-[(4-chlorophenyl)thio]-6-fluorophenyl}-N-(4-hydroxybutyl)acrylamide;
(2E)-N-(4-hydroxybutyl)-3-{2-[(4-methoxyphenyl)thio]phenyl}acrylamide;
(2E)-3-{2-[(2,4-dimethylphenyl)thio]phenyl}-N-(5-hydroxy-1,5-dimethylhexyl)acrylamide; and
(2E)-3-{2-[(4-chlorophenyl)thio]-5-fluorophenyl}-N-(4-hydroxybutyl)acrylamide.
